SEQUENCE LISTING (26 sequences)

SEQ ID NO: 1 - protein, 117 aa, unidentified
EVQLQQSGPELVKPGASVKMSCKASGYTFANHVMHWVKQKPGQGLEWIGYIYPYNDGTKYNEKFKGKATLTSDKSSSTAYMELSSLASEDSAVYYCARNNPAWFAYWGQGTLVTVSA

SEQ ID NO: 2 - protein, 117 aa, unidentified; identical to SEQ ID NO: 1

SEQ ID NO: 3 - protein, 115 aa, unidentified
EVQLQQSGPELVKPGASVKMSCKASGYTFANHVMHWVKQKPGQGLEWIGYIYPYNDGTKYNEKFKGKATLTSDKSSSTAYMELSSLASEDSAVYYCASNYYFDYWGQGTTLTVSS

SEQ ID NO: 4 - protein, 115 aa, unidentified; identical to SEQ ID NO: 3

SEQ ID NO: 5 - protein, 447 aa, unidentified
EVQLQQSGPELVKPGASVKMSCKASGYTFANHVMHWVKQKPGQGLEWIGYIYPYNDGTKYNEKFKGKATLTSDKSSSTAYMELSSLASEDSAVYYCARNNPAWFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 6 - protein, 446 aa, unidentified
EVQLQQSGPELVKPGASVKMSCKASGYTFANHVMHWVKQKPGQGLEWIGYIYPYNDGTKYNEKFKGKATLTSDKSSSTAYMELSSLASEDSAVYYCARNNPAWFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 445 aa, unidentified
EVQLQQSGPELVKPGASVKMSCKASGYTFANHVMHWVKQKPGQGLEWIGYIYPYNDGTKYNEKFKGKATLTSDKSSSTAYMELSSLASEDSAVYYCASNYYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 8 - protein, 444 aa, unidentified
EVQLQQSGPELVKPGASVKMSCKASGYTFANHVMHWVKQKPGQGLEWIGYIYPYNDGTKYNEKFKGKATLTSDKSSSTAYMELSSLASEDSAVYYCASNYYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 113 aa, unidentified
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSYTFGGGTKLEIKR

SEQ ID NO: 10 - protein, 107 aa, unidentified
QIVLTQSPAIMSASPGEKVTMTCSASSSVSYMHWYQQKSGTSPKRWIYDTSKLASGVPARFSGSGSGTSYSLTISSMEAEDAATYYCQQWSSNPYTFGGGTKLEIKR

SEQ ID NO: 11 - protein, 219 aa, unidentified
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHATSTSPIVKSFNRNEC

SEQ ID NO: 12 - protein, 213 aa, unidentified
QIVLTQSPAIMSASPGEKVTMTCSASSSVSYMHWYQQKSGTSPKRWIYDTSKLASGVPARFSGSGSGTSYSLTISSMEAEDAATYYCQQWSSNPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - protein, 97 aa, unidentified
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 14 - protein, 126 aa, unidentified
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPK

SEQ ID NO: 15 - protein, 107 aa, unidentified
GSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 16 - protein, 97 aa, unidentified; identical to SEQ ID NO: 13

SEQ ID NO: 17 - protein, 125 aa, unidentified
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 18 - protein, 107 aa, unidentified
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 97 aa, unidentified; identical to SEQ ID NO: 13

SEQ ID NO: 20 - protein, 126 aa, unidentified; identical to SEQ ID NO: 14

SEQ ID NO: 21 - protein, 107 aa, unidentified; identical to SEQ ID NO: 15

SEQ ID NO: 22 - protein, 97 aa, unidentified; identical to SEQ ID NO: 13

SEQ ID NO: 23 - protein, 125 aa, unidentified; identical to SEQ ID NO: 17

SEQ ID NO: 24 - protein, 107 aa, unidentified; identical to SEQ ID NO: 18

SEQ ID NO: 25 - protein, 106 aa, unidentified
ADAAPTVSIFPPSSEQLTSGGASVVCFLNNFYPKDINVKWKIDGSERQNGVLNSWTDQDSKDSTYSMSSTLTLTKDEYERHNSYTCEATHATSTSPIVKSFNRNEC

SEQ ID NO: 26 - protein, 106 aa, unidentified
ADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2025/0163187 A1
(43) Pub. Date: May 22, 2025

(54) COMPOSITIONS AND METHODS FOR DETECTING dsRNA

(71) Applicant: ModernaTX, Inc., Cambridge, MA (US)

(72) Inventors: Kristian LINK, Sharon, MA (US); Tao JIANG, Quincy, MA (US); Graham FARRINGTON, Acton, MA (US); Huijuan LI, Wayland, MA (US)

(21) Appl. No.: 19/033,264

(22) Filed: Jan. 21, 2025

Related U.S. Application Data
(63) Continuation of application No. PCT/US2023/028350, filed on Jul. 21, 2023.
(60) Provisional application No. 63/391,376, filed on Jul. 22, 2022.

(51) Int. Cl. C07K 16/44; G01N 33/53

(52) U.S. Cl. CPC C07K 16/44; G01N 33/5308; C07K 2317/565; C07K 2317/567

(57) ABSTRACT

The present disclosure relates generally to antibodies and antigen-binding fragments that bind to double stranded RNA (dsRNA) and technologies for detection and/or quantification of dsRNA.

### CROSS-REFERENCE TO RELATED APPLICATION

This application is a continuation of PCT/US2023/028350 filed Jul. 21, 2023, which application claims priority to U.S. Provisional Application Ser. No. 63/391,376 filed Jul. 22, 2022, the disclosures of both of which are incorporated herein by reference.

### REFERENCE TO AN ELECTRONIC SEQUENCE LISTING

The contents of the electronic sequence listing (131986-0901.xml; Size: 27,630 bytes; and Date of Creation: Jan. 21, 2025) is herein incorporated by reference in its entirety.

### BACKGROUND

The following description of the background of the present technology is provided simply as an aid in understanding the present technology and is not admitted to describe or constitute prior art to the present technology.

The use of messenger RNA (e.g., mRNA) is increasingly important in biomedical research and clinically as a therapeutic modality. Detection and/or quantification of dsRNA has been particularly challenging, due at least in part to the limited availability of dsRNA-specific antibodies and antigen-binding polypeptides and the difficulty of developing such antibodies or antigen-binding polypeptides that are not sequence-dependent.

Accordingly, there remains a need for efficient and sensitive technologies for detection and quantification of dsRNA, in particular, in samples of IVT RNAs.

### SUMMARY OF THE INVENTION

The present disclosure provides, among other things, antibodies and antigen-binding fragments that specifically bind to double stranded RNA (dsRNA) in a sequence-independent manner. Such antibodies and antigen-binding fragments are useful in the detection and quantification of dsRNA and can be incorporated into various detection methods, including, for example, Enzyme-Linked Immunosorbent Assays (ELISAs).

In one aspect, the present disclosure provides antibodies or antigen-binding fragments thereof that binds to double stranded RNA (dsRNA), wherein the antibody or antigen-binding fragment thereof comprises: (i) complementarity determining regions (CDRs) of a heavy chain variable domain comprising an amino acid sequence selected from SEQ ID NO: 1, SEQ ID NO: 2, SEQ ID NO: 3, and SEQ ID NO: 4; and (ii) CDRs of a light chain variable domain comprising an amino acid sequence selected from SEQ ID NO: 9 and SEQ ID NO: 10.

In some embodiments, the antibody or antigen-binding fragment thereof comprises the CDRs of the heavy chain variable domain comprising the amino acid sequence of SEQ ID NO: 1 and the CDRs of the light chain variable domain comprising the amino acid sequence of SEQ ID NO: 9.

In some embodiments, the antibody or antigen-binding fragment thereof comprises the CDRs of the heavy chain variable domain comprising the amino acid sequence of SEQ ID NO: 2 and the CDRs of the light chain variable domain comprising the amino acid sequence of SEQ ID NO: 9.

In some embodiments, the antibody or antigen-binding fragment thereof comprises the CDRs of the heavy chain variable domain comprising the amino acid sequence of SEQ ID NO: 3 and the CDRs of the light chain variable domain comprising the amino acid sequence of SEQ ID NO: 10.

In some embodiments, the antibody or antigen-binding fragment thereof comprises the CDRs of the heavy chain variable domain comprising the amino acid sequence of SEQ ID NO: 4 and the CDRs of the light chain variable domain comprising the amino acid sequence of SEQ ID NO: 10. In some embodiments, the antibody or antigen-binding fragment thereof comprises mammalian framework regions and mammalian constant regions. In some embodiments, the mammalian framework regions and mammalian constant regions are human or murine. In some embodiments, the antibody is an IgG antibody.

In some embodiments, the antibody or antigen-binding fragment thereof comprises a heavy chain variable domain comprising an amino acid sequence selected from the group consisting of SEQ ID NO: 1, SEQ ID NO: 2, SEQ ID NO: 3, and SEQ ID NO: 4.

In some embodiments, the antibody or antigen-binding fragment thereof comprises a light chain variable domain comprising an amino acid sequence selected from the group consisting of SEQ ID NO: 9 and SEQ ID NO: 10.

In some embodiments, the heavy chain variable domain comprises the amino acid sequence of SEQ ID NO: 1, and the light chain variable domain comprises the amino acid sequence of SEQ ID NO: 9. In some embodiments, the heavy chain variable domain comprises the amino acid sequence of SEQ ID NO: 2, and the light chain variable domain comprises the amino acid sequence of SEQ ID NO: 9. In some embodiments, the heavy chain variable domain comprises the amino acid sequence of SEQ ID NO: 3, and the light chain variable domain comprises the amino acid sequence of SEQ ID NO: 10. In some embodiments, the heavy chain variable domain comprises the amino acid sequence of SEQ ID NO: 4, and the light chain variable domain comprises the amino acid sequence of SEQ ID NO: 10. In some embodiments, the antibody or antigen-binding fragment thereof comprises mammalian constant regions. In some embodiments, the mammalian framework regions and mammalian constant regions are human or murine. In some embodiments, the antibody is an IgG antibody.

In some embodiments, the antibody or antigen-binding fragment thereof comprises a heavy chain comprising an amino acid sequence having at least 80% identity to the amino acid sequence selected from the group consisting of SEQ ID NO: 5, SEQ ID NO:6, SEQ ID NO: 7, and SEQ ID NO: 8. In some embodiments, the antibody or antigen-binding fragment thereof comprises a heavy chain comprising an amino acid sequence selected from the group consisting of SEQ ID NO: 5, SEQ ID NO:6, SEQ ID NO: 7, and SEQ ID NO: 8.

In some embodiments, the antibody or antigen-binding fragment thereof comprises a light chain comprising an amino acid sequence having at least 80% identity to the amino acid sequence selected from the group consisting of SEQ ID NO: 11 and SEQ ID NO: 12. In some embodiments, the antibody or antigen-binding fragment thereof comprises a light chain comprising an amino acid sequence selected from the group consisting of SEQ ID NO: 11 and SEQ ID NO: 12.

In some embodiments, the heavy chain comprises the amino acid sequence having at least 80% identity to the amino acid sequence of SEQ ID NO: 5, and the light chain comprises the amino acid sequence having at least 80% identity to the amino acid sequence of SEQ ID NO: 11. In some embodiments, the heavy chain comprises the amino acid sequence of SEQ ID NO: 5, and the light chain comprises the amino acid sequence of SEQ ID NO: 11. In some embodiments, the heavy chain comprises the amino acid sequence having at least 80% identity to the amino acid sequence of SEQ ID NO: 6, and the light chain comprises the amino acid sequence having at least 80% identity to the amino acid sequence of SEQ ID NO: 11. In some embodiments, the heavy chain comprises the amino acid sequence of SEQ ID NO: 6, and the light chain comprises the amino acid sequence of SEQ ID NO: 11. In some embodiments, the heavy chain comprises the amino acid sequence having at least 80% identity to the amino acid sequence of SEQ ID NO: 7, and the light chain comprises the amino acid sequence having at least 80% identity to the amino acid sequence of SEQ ID NO: 12. In some embodiments, the heavy chain comprises the amino acid sequence of SEQ ID NO: 7, and the light chain comprises the amino acid sequence of SEQ ID NO: 12. In some embodiments, the heavy chain comprises the amino acid sequence having at least 80% identity to the amino acid sequence of SEQ ID NO: 8, and the light chain comprises the amino acid sequence having at least 80% identity to the amino acid sequence of SEQ ID NO: 12. In some embodiments, the heavy chain comprises the amino acid sequence of SEQ ID NO: 8, and the light chain comprises the amino acid sequence of SEQ ID NO: 12.

In one aspect, the present disclosure provides methods for detecting or quantifying double stranded RNA (dsRNA) in a sample comprising: (i) contacting the sample with a capture antibody selected from an anti-dsRNA antibody or antigen-binding fragment thereof, thereby producing a first complex comprising the dsRNA and the capture antibody, wherein the capture antibody is immobilized on a solid substrate; (ii) contacting the first complex with a detection antibody selected from an anti-dsRNA antibody or antigen-binding fragment thereof, thereby producing a second complex comprising the dsRNA, the capture antibody, and the detection antibody; (iii) optionally contacting the second complex with a secondary antibody that binds to the detection antibody; wherein either of the detection antibody or the secondary antibody comprises a label capable of producing a signal; and (iv) measuring the signal.

In some embodiments, the sample comprises drug substance of a RNA-based therapeutic.

In some embodiments, the capture antibody and the detection antibody are from different species. In some embodiments, the capture antibody and the detection antibody are from the same species. In some embodiments, the capture antibody and the detection antibody are different isotypes or subclasses. In some embodiments, the different subclasses are (i) selected from human IgG1, IgG2, IgG3, and IgG4; (ii) selected from mouse IgG1, IgG2A, IgG2B, IgG2C, and IgG3; or (iii) selected from rat IgG1, IgG2A, IgG2B, and IgG2C.

In some embodiments, the capture antibody and the anti-dsRNA detection antibody comprise the same CDRs.

In some embodiments, the capture antibody, detection antibody, or both is an IgG. In some embodiments, the detection antibody is not an IgM.

In some embodiments, the detection antibody and the secondary antibody are from different species.

In some embodiments, the label comprises horseradish peroxidase (HRP). In some embodiments, the label comprises a fluorescent dye.

In some embodiments, the methods may further comprise washing the solid substrate between each of (i), (ii), (iii), (iv), or any combination thereof.

In some embodiments, the methods may further comprise a blocking step before (i).

In some embodiments, the capture antibody comprises (i) heavy chain CDRs of SEQ ID NO: 3 and light chain CDRs of SEQ ID NO: 9, or (ii) heavy chain CDRs of SEQ ID NO: 4 and light chain CDRs of SEQ ID NO: 10.

In some embodiments, the detection antibody comprises (i) heavy chain CDRs of SEQ ID NO: 3 and light chain CDRs of SEQ ID NO: 9, or (ii) heavy chain CDRs of SEQ ID NO: 4 and light chain CDRs of SEQ ID NO: 10.

In some embodiments, the capture antibody comprises (i) a variable heavy chain comprising an amino acid sequence of SEQ ID NO: 3 and a variable light chain comprising an amino acid sequence of SEQ ID NO: 9, or (ii) a variable heavy chain comprising an amino acid sequence of SEQ ID NO: 4 and a variable light chain comprising an amino acid sequence of SEQ ID NO: 10.

In some embodiments, the detection antibody comprises (i) a variable heavy chain comprising an amino acid sequence of SEQ ID NO: 3 and a variable light chain comprising an amino acid sequence of SEQ ID NO: 9, or (ii) a variable heavy chain comprising an amino acid sequence of SEQ ID NO: 4 and a variable light chain comprising an amino acid sequence of SEQ ID NO: 10.

In one aspect, the present disclosure provides methods for detecting or quantifying double stranded RNA (dsRNA) in a sample comprising: (i) contacting the sample with a capture antibody that binds to dsRNA, thereby producing a first complex comprising the dsRNA and the capture antibody, wherein the capture antibody is immobilized on a solid substrate; (ii) contacting the first complex with a detection antibody that binds to dsRNA, thereby producing a second complex comprising the dsRNA, the capture antibody, and the detection antibody; (iii) optionally contacting the second complex with a secondary antibody that binds to the detection antibody; wherein either of the detection antibody or the secondary antibody comprises a label capable of producing a signal; and (iv) measuring a signal; wherein the capture antibody and the detection antibody comprise the same CDRs but comprise different Fc domains or constant regions.

In some embodiments, the sample comprises drug substance of a RNA-based therapeutic.

In some embodiments, the different Fc domains or constant regions are from different species. In some embodiments, the capture antibody is an IgG. In some embodiments, the detection antibody is an IgG. In some embodiments, both the capture antibody and the detection antibody are IgGs. In some embodiments, the capture antibody and the detection antibody are from the same species. In some embodiments, the capture antibody and the detection antibody are different isotypes or subclasses. In some embodiments, the different subclasses are (i) selected from human IgG1, IgG2, IgG3, and IgG4; (ii) selected from mouse IgG1, IgG2A, IgG2B, IgG2C, and IgG3; or (iii) selected from rat IgG1, IgG2A, IgG2B, and IgG2C.

In some embodiments, the capture antibody comprises (i) heavy chain CDRs of SEQ ID NO: 3 and light chain CDRs of SEQ ID NO: 9, or (ii) heavy chain CDRs of SEQ ID NO: 4 and light chain CDRs of SEQ ID NO: 10.

In some embodiments, the detection antibody comprises (i) heavy chain CDRs of SEQ ID NO: 3 and light chain CDRs of SEQ ID NO: 9, or (ii) heavy chain CDRs of SEQ ID NO: 4 and light chain CDRs of SEQ ID NO: 10.

In some embodiments, the detection antibody and the secondary antibody are from different species.

In some embodiments, the label comprises horseradish peroxidase (HRP).

In some embodiments, the label comprises a fluorescent dye.

In some embodiments, the methods may further comprise washing the solid substrate between each of (i), (ii), (iii), (iv), or any combination thereof.

In some embodiments, the methods may further comprise a blocking step before (i).

In one aspect, the present disclosure provides kits comprising a capture antibody that binds to dsRNA antibody and a detection antibody that binds to dsRNA.

In some embodiments, the capture antibody and the detection antibody comprise Fc domains or constant regions that are from different species.

In some embodiments, the capture antibody and the detection antibody comprise the same CDRs.

In some embodiments, the capture antibody, detection antibody, or both is an IgG.

In some embodiments, the capture antibody comprises (i) heavy chain CDRs of SEQ ID NO: 3 and light chain CDRs of SEQ ID NO: 9, or (ii) heavy chain CDRs of SEQ ID NO: 4 and light chain CDRs of SEQ ID NO: 10.

In some embodiments, the detection antibody comprises (i) heavy chain CDRs of SEQ ID NO: 3 and light chain CDRs of SEQ ID NO: 9, or (ii) heavy chain CDRs of SEQ ID NO: 4 and light chain CDRs of SEQ ID NO: 10.

In some embodiments, the detection antibody comprises a detectable label. In some embodiments, the detectable label is HRP or a fluorophore.

In some embodiments, the kit may further comprise a secondary antibody that binds to the detection antibody and which comprises a detectable label.

In some embodiments, the kit may further comprise one or more reagents for detecting dsRNA. In some embodiments, the one or more reagents includes a blocking buffer. In some embodiments, the one or more reagents includes a wash buffer. In some embodiments, the one or more reagents includes a positive control.

The foregoing general description and following detailed description are exemplary and explanatory and are intended to provide further explanation of the invention.

### BRIEF DESCRIPTION OF THE DRAWINGS

FIG. 1A-1D demonstrate detection of dsRNA using Antibody 1 (Ab1), Antibody 2 (Ab2), Antibody 3 (Ab3), and Antibody 4 (Ab4), as capture antibodies. FIG. 1A shows mean absorption of Ab1, Ab2, Ab3, and Ab4 at 650 nm. FIG. 1B-1D demonstrates that Ab1 and Ab2 did not specifically bind to dsRNA, while Ab3 and Ab4 specifically bound to dsRNA with an EC₅₀ of 8.362 and 8.041, respectively, while a first control anti-dsRNA antibody, Antibody 5 (Ab5), specifically bound dsRNA with an EC₅₀ of 10.59.

### DETAILED DESCRIPTION

Detection and quantification of dsRNA has been challenging. Currently, a procedure known as a dot blot assay (or “dot blotting analysis”) is the standard method of quantifying RNA, but dot blots, while simple and fast, are notoriously prone to low sensitivity and high levels of background. The traditional dot blotting procedure includes two key steps of sample fixation onto membrane and final autoradiography. The performance of dot blotting depends on the quality of fixing and the selection of membrane besides other factors. The commonly used method of fixation is ultraviolet (UV) fixing, and the commonly used membranes are nitrocellulose (NC) membrane and nylon (NL) membrane. NC membrane is a generally used good supporting material for many methods because of its low background. However, it has very low ability to bind with nucleic acids, such as RNA. NL membrane is also a commonly used supporting material with relatively high binding capacity to biological molecules. However, it shows a very high background.

Detection and quantification of other biological macromolecules (e.g., proteins, carbohydrates, etc.) are often performed using antibody-based methods, but the same does not hold true for RNA. This may be due, at least in part, to the limited availability of RNA-specific antibodies and antigen-binding fragments that specifically bind dsRNA in a sequence-independent manner. Indeed, sequence-independent binding is paramount to being able to develop a broad-spectrum platform that could be used to detect or quantify any RNA, rather than being limited only to particular RNA sequences.

The present disclosure provides, among other things, antibodies and antigen-binding fragments that specifically bind or are capable of specifically binding double stranded RNA (dsRNA), compositions and kits including the same, and uses thereof, including methods for detecting and/or quantifying dsRNA. The disclosed methods and uses are highly sensitive and may provide improved sensitivity over currently utilized technologies, such as dot blots.

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

### Definitions

As used in the specification and claims, the singular form “a,” “an,” and “the” include singular and plural references unless the context clearly dictates otherwise. For example, the term “a cell” includes a single cell as well as a plurality of cells, including mixtures thereof.

As used herein, the term “affinity” refers to the characteristics of a binding interaction between a binding moiety and a target and that indicates the strength of the binding interaction. In some embodiments, the measure of affinity is expressed as a dissociation constant (K_{D}). In some embodiments, a binding moiety has a high affinity for a target (e.g., a K_{D} of less than about 10⁻⁷ M, less than about 10⁻⁸ M, or less than about 10⁻⁹ M). In some embodiments, a binding moiety has a low affinity for a target (e.g., a K_{D} of higher than about 10⁻⁷ M, higher than about 10⁻⁶ M, higher than about 10⁻⁵ M, or higher than about 104 M).

As used herein, the term “approximately” or “about” means plus or minus 10% as well as the specified number. For example, “about 10” should be understood as both “10” and “9-11”.

As used herein, an “antibody” or “antibodies” include immunoglobulin molecules that contain an antigen binding site. Immunoglobulin molecules can be of any type (e.g., IgG, IgE, IgM, IgD, IgA, and IgY), class (e.g., IgG1, IgG2, IgG3, IgG4, IgA1, and IgA2), or subclass. In certain embodiments, an antibody is a glycoprotein comprising at least two heavy (H) chains and two light (L) chains inter-connected by disulfide bonds. Each heavy chain is comprised of a heavy chain variable region (abbreviated herein as VH) and a heavy chain constant (CH) region. The heavy chain constant region is comprised of three domains, CH1, CH2, and CH3. Each light chain is comprised of a light chain variable region (abbreviated herein as VL) and a light chain constant CL region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl q) of the classical complement system.

An “isolated antibody” or “isolated antigen binding protein” is one which has been identified and separated and/or recovered from a component of its natural environment. “Synthetic antibodies” or “recombinant antibodies” are generally generated using recombinant technology or using peptide synthetic techniques known to those of skill in the art. Antibodies and antibody fragments can be wholly or partially derived from mammals (e.g., humans, non-human primates, goats, guinea pigs, hamsters, horses, mice, rats, rabbits and sheep) or non-mammalian antibody producing animals (e.g., chickens, ducks, geese, snakes, and urodele amphibians). The antibodies and antibody fragments can be produced in animals or produced outside of animals, such as from yeast or phage (e.g., as a single antibody or antibody fragment or as part of an antibody library). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules. These are known as single chain Fv (scFv); see e.g., Bird et al., Science 242:423-426 (1988); and Huston et al., Proc. Natl. Acad. Sci.85:5879-5883 (1988). These antibody fragments are obtained using conventional techniques known to those of ordinary skill in the art, and the fragments are screened for utility in the same manner as are intact antibodies.

As used herein, the term “fragment,” when used in the context of referring to an antibody, means a portion or portions of antibody molecules that retain antigen-binding ability. Such fragments are also well known in the art and are regularly employed both in vitro and in vivo. Examples of binding fragments include (i) Fab fragments (monovalent fragments consisting of the V_{L}, V_{H}, C_{L} and C_{H1} domains); (ii) F(ab′)₂ fragments (bivalent fragment comprising two Fab fragments linked by a disulfide bridge at the hinge region); (iii) Fd fragments (comprising the V_{H} and C_{H1} domains); (iv) Fv fragments (comprising the V_{L} and V_{H} domains of a single arm of an antibody), (v) dAb fragments (comprising a V_{H} domain); and (vi) isolated complementarity determining regions (CDR), e.g., V_{H} CDR3. Other examples include single chain Fvs (scFvs), single domain antibodies (e.g., nanobodies and single domain camelid antibodies), VNAR fragments, Bi-specific T-cell engager (BiTE) antibodies, minibodies, disulfide-linked Fvs (sdFv), and anti-idiotypic (anti-Id) antibodies, intrabodies, and fusion polypeptides. For the purposes of the present disclosure, the terms “fragment,” “antigen-binding fragment,” and “antigen-binding polypeptide” may be used interchangeably.

As used herein, the term “binding” typically refers to a non-covalent association between or among two or more entities. “Direct” binding involves physical contact between entities or moieties; “indirect” binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system such as a cell).

As used herein, a “binding moiety” is any molecule or part of a molecule capable of specifically binding a target, e.g., a target of interest. Binding moieties include, e.g., antibodies and antigen binding fragments thereof. As used herein, an “antigen-binding polypeptide” is any polypeptide including a binding moiety, which antigen-binding polypeptide can be, in certain non-limiting examples, an antibody or a fragment thereof. An “antigen-binding polypeptide” can be any of, without limitation, a heavy chain antibody, light chain antibody, LRR-based antibody, other protein scaffold with antibody-like properties, or other immunological binding moiety known in the art, including, e.g., a four-chain immunoglobulin, imunoadhesin, diabody, dsFv, diabody, triabody, tetrabody, minibody, maxibody, TandAb, single chain antibody, heavy chain antibody, single domain heavy chain antibody, particular HCDR, particular LCDR, heavy chain variable domain, light chain variable domain, DVD, BiTe, scFv, scAb, Fab, Fab′, Fab2, Fab3, F(ab′)2, Fd, Fd′, Fv or the like, or any combination thereof.

As used herein, the term “CDR-grafted antibody” refers to an antibody whose amino acid sequence comprises heavy and light chain variable region sequences of a first species but in which the sequences of one or more of the CDR regions of VH and/or VL are replaced with CDR sequences of another species, such as (to provide just one example) an antibody having murine VH and VL regions in which one or more of the murine CDRs (e.g., CDR3) has been replaced with human CDR sequences. Likewise, a “CDR-grafted antibody” encompasses, as an example, an antibody having human VH and VL regions in which one or more of the human CDRs (e.g., CDR3) has been replaced with mouse CDR sequences.

As used herein, the term “constant region” refers to a polypeptide that corresponds to, or is derived from, one or more constant region immunoglobulin domains of an antibody. A constant region can include any or all of the following immunoglobulin domains: a CH1 domain, a hinge region, a CH2 domain, a CH3 domain (derived from an IgA, IgD, IgG, IgE, or IgM), and a CH4 domain (derived from an IgE or IgM).

As used herein, the term “epitope” includes any moiety that is specifically recognized by an antibody or antigen-binding fragment, or a binding moiety thereof.

As used herein, the term “humanized” can refer to an antibody or antigen-binding polypeptide having an amino acid sequence that includes VH and VL region sequences from a reference antibody raised in a non-human species (e.g., a mouse), but also includes modifications in those sequences relative to the reference antibody intended to render them more “human-like,” i.e., more similar to human germline variable sequences. In some embodiments, a “humanized” antibody or antigen-binding polypeptide is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody or antigen-binding polypeptide can be an antibody or antigen-binding polypeptide in which substantially all of at least one, and typically two, variable domains (Fab, Fab′, F(ab′)2, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody or antigen-binding polypeptide includes at least a portion of an immunoglobulin constant region (Fc), typically that of a human immunoglobulin constant region. In some embodiments, a humanized antibody or antigen-binding polypeptide contains both the light chain as well as at least the variable domain of a heavy chain. A humanized antibody or antigen-binding polypeptide can include a CH1, hinge, CH2, CH3, and, optionally, a CH4 region of a heavy chain constant region. In some embodiments, a humanized antibody or antigen-binding polypeptide only contains a humanized VL region. In some embodiments, a humanized antibody or antigen-binding polypeptide only contains a humanized VH region. In some certain embodiments, a humanized antibody or antigen-binding polypeptide contains humanized VH and VL regions.

As used herein, the terms “improved”, “increased”, or “reduced”, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, a “reference” entity, system, amount, set of conditions, etc., is one against which a test entity, system, amount, set of conditions, etc. is compared as described herein. For example, in some embodiments, a “reference” antibody or antigen-binding polypeptide is a control antibody or antigen-binding polypeptide, e.g., an antibody or antigen-binding polypeptide that is not described herein. In some embodiments, a reference or control is tested and/or determined simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, “selective binding,” “selectively binds,” “specific binding,” or “specifically binds” refers, with respect to an antibody/binding moiety and a target, preferential association of the antibody/binding moiety to a target and not to an entity that is not the target. A certain degree of non-specific binding may occur between a binding moiety and a non-target.

As used herein, a “target” or “target of interest” is any molecule specifically bound by an antibody/binding moiety.

As used herein, the term “vector” refers to a recipient nucleic acid molecule modified to include or incorporate a provided nucleic acid sequence. One type of vector is a “plasmid” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### Anti-RNA Antibodies

The disclosed antibodies and antigen-binding fragments are unique in their ability to bind to RNA (e.g., double stranded or dsRNA) in a sequence-independent manner, meaning that the antibodies and fragments can bind to various RNA molecules regardless of the nucleotides making up the molecule (i.e., regardless of the sequence). Without being bound by theory, it is believed that the disclosed antibodies and antigen-binding fragments may bind to the phosphate backbone of the RNA structure.

This sequence-independent binding allows the claimed antibodies to be used in generalized methods of detecting and quantifying RNA, as disclosed in more detail herein. For example, the disclosed antibodies or fragments could be used to detect/quantify the amount of RNA in two different batches of therapeutic mRNAs, even if the mRNAs are different sequences. Thus, the disclosed antibodies an methods provide a broad-spectrum platform for detection and quantification that can facilitate quality control in the production of any RNA product, such as therapeutic RNAs that can include, but are not limited to, messenger RNAs (mRNAs), micro RNAs (miRNAs), short interfering RNAs (siRNAs), antisense oligonucleotides (ASOs), short hairpin RNAs (shRNAs), heterodimeric oligonucleotides (HDOs), and any other RNA-based molecules.

An antibody can be an immunoglobulin molecule of four polypeptide chains, e.g., two heavy chains (HCs) and two light chains (LCs). An antigen-binding fragment can be or comprise one or more of a heavy chain, light chain, heavy chain variable domain, light chain variable domain, or any of one or more portions thereof as described herein or otherwise known in the art. A heavy chain can comprise a heavy chain variable domain and a heavy chain constant domain. A heavy chain constant domain can comprise CH1, hinge, CH2, CH3, and in some instances, CH4 regions. A light chain can comprise a light chain variable domain and a light chain constant domain (CL). A heavy chain variable domain of a heavy chain and a light chain variable domain of a light chain can typically be further subdivided into regions of variability, referred to as complementarity determining regions (CDRs), interspersed with regions that are more conserved, referred to as framework regions (FR). Framework regions are not typically determinative of antigen-binding. Framework regions of antibodies and antigen-binding polypeptides of the present disclosure can be any framework region disclosed herein or known in the art. Such heavy chain and light chain variable domains can each include three CDRs and four framework regions, arranged from amino-terminus to carboxyl-terminus in the following order: FR1, CDR1, FR2, CDR2, FR3, CDR3, FR4, one or more of which can have a sequence as described herein.

Certain systems have been established in the art for defining CDR boundaries (e.g., the Kabat system and the Chothia system). Those of skill in the art will be aware that selection of a CDR identifying system (e.g., the Kabat system or Chothia system) will impact the amino acid sequence of identified CDRs (and accordingly the sequences of CDR-encoding nucleic acids). Thus, the skilled artisan will appreciate that while the present disclosure may identify CDRs according to a particular system, the present disclosure encompasses those CDRs as may be identified by any alternative system.

The present disclosure, among other things, provides anti-dsRNA antibodies or antigen-binding fragments that may be used, for example, in the detection and/or quantitation of dsRNA. In some embodiments, an anti-dsRNA antibody or fragment thereof described herein may be polyclonal, monoclonal, chimeric, human, partially or fully humanized, CDR-grafted and/or recombinant. In particular, a disclosed antibody may comprise CDRs from one species (e.g., a mouse or rat), which are humanized or native, while the remainder of the antibody (e.g., the framework and constant regions) are from a different species (e.g., a human). In some embodiments, the framework regions, constant regions, or both are mammalian. In some embodiments, the framework regions, constant regions, or both are human. In some embodiments, the framework regions, constant regions, or both are murine.

Further, the disclosed anti-dsRNA antibodies may be any isotype, including IgA, IgD, IgE, IgG, or IgM, including subclasses, which include, for example and without limitation, IgA1, IgA2, IgG1, IgG2, IgG3, IgG4. In some embodiments, the disclosed anti-dsRNA antibodies are IgG. In some embodiments, the disclosed anti-dsRNA antibodies are not IgM.

An anti-dsRNA antibody or antigen-binding fragment of the present disclosure may comprise a combination of CDR sequences, variable domain sequences, or full-length heavy and light chain sequences, as disclosed herein. Exemplary antibody sequences are provided below. In some embodiments, an anti-dsRNA antibody may comprise four polypeptides: two identical copies of a heavy chain and two identical copies of a light chain (e.g., as described herein). In some embodiments, each heavy chain comprises one N-terminal variable domain (VH) and three C-terminal constant (CH1, CH2, CH3) domains, and each light chain comprises one N-terminal variable domain (VL) and one C-terminal constant domain (CL). In some embodiments, the variable domains of each pair of light and heavy chains form the antigen binding site of an antibody.

In some embodiments, an anti-dsRNA antibody or antigen-binding polypeptide comprises one or two heavy chain variable domains and one or more light chain variable domains. In some embodiments, an anti-dsRNA antibody or antigen-binding polypeptide comprises a first anti-dsRNA heavy chain variable domain, and a second anti-dsRNA heavy chain variable domain that is the same or different from the first anti-dsRNA heavy chain variable domain, and an anti-dsRNA light chain variable domain. In some embodiments, an anti-dsRNA antibody or antigen-binding polypeptide comprises a first anti-dsRNA light chain variable domain, a second anti-dsRNA light chain variable domain that is the same or different from the first anti-dsRNA light chain variable domain, and an anti-dsRNA heavy chain variable domain. In certain embodiments, an anti-dsRNA antibody or antigen-binding polypeptide comprises a first anti-dsRNA heavy chain variable domain, a second anti-dsRNA heavy chain variable domain that is same as or different from the first anti-dsRNA heavy chain variable domain, a first anti-dsRNA light chain variable domain, and a second anti-dsRNA light chain variable domain that is same as or different from the first anti-dsRNA light chain variable domain.

In some embodiments, anti-dsRNA antibodies or antigen-binding polypeptides comprise one or two heavy chains and one or two light chains. In certain embodiments, an anti-dsRNA antibody or antigen-binding polypeptide comprises a first anti-dsRNA heavy chain, a second anti-dsRNA heavy chain that is same as or different from the first anti-dsRNA heavy chain, and an anti-dsRNA light chain. In some embodiments, an anti-dsRNA antibody or antigen-binding polypeptide comprises a first anti-dsRNA light chain, a second anti-dsRNA light chain that is same as or different from the first anti-dsRNA light chain, and an anti-dsRNA heavy chain. In some embodiments, an anti-dsRNA antibody or antigen-binding polypeptide comprises a first anti-dsRNA heavy chain, a second anti-dsRNA heavy chain that is same as or different from the first anti-dsRNA heavy chain, a first anti-dsRNA light chain, and a second anti-dsRNA light chain that is same as or different from the first anti-dsRNA light chain.

Anti-dsRNA antibodies or antigen-binding polypeptides described herein can include a hinge region (e.g., a short sequence of a heavy chain that links the Fab region to the Fc region). In some embodiments, a hinge region of the disclosed antibodies and antigen-binding polypeptides may be modified by replacing one or more cysteine residues with, for example, serine residues, to prevent dimerization. See, e.g., U.S. Patent Application Publication 2003/0118592; U.S. Patent Application Publication U.S. 2003/0133939. Additionally, in some embodiments, the disclosed antibodies or antigen-binding polypeptides may comprise other mutations. Such modifications may alter the binding of the disclosed antibodies and antigen-binding polypeptides, and in some embodiments, the antibody or antigen-binding polypeptide may be modified to be more stable. When determining the number of the residue, the Kabat numbering of residues may be determined for a given antibody by alignment at regions of homology of the sequence of the antibody with a “standard” Kabat numbered sequence.

Anti-dsRNA antibodies or antigen-binding fragments described here may be obtained by methods known in the art. For example, polyclonal antibodies may be obtained by immunizing a selected animal with an antigen (e.g., dsRNA), collecting serum from the animal, and isolating and/or purifying antibodies from the serum. Monoclonal antibodies may be obtained, for example, by fusing antibody-producing cells with immortalized cells to obtain a hybridoma, and/or by generating mAbs from mRNA extracted from bone marrow and spleen cells of immunized animals using combinatorial antibody library technology. Recombinant antibodies may be obtained, for example, by using phage or yeast display technologies and/or expressing or co-expressing antibody polypeptides.

### A. Exemplary Heavy Chain Complementary Determining Regions (HCDRs)

The present disclosure, among other things, provides anti-dsRNA HCDR sequences. In some embodiments, an anti-dsRNA antibody or antigen-binding fragment comprises one or more of an anti-dsRNA HCDR1, an anti-dsRNA HCDR2, and an anti-dsRNA HCDR3. In some embodiments, an anti-dsRNA antibody or antigen-binding polypeptide comprises CDRs of a heavy chain variable domain of any one of SEQ ID NO: 1, SEQ ID NO: 2, SEQ ID NO: 3, or SEQ ID NO: 4.

In some embodiments, an anti-dsRNA antibody or antigen-binding polypeptide described herein comprises anti-dsRNA HCDR1, anti-dsRNA HCDR2, and an anti-dsRNA HCDR3 having at least 70% sequence identity, at least 75% sequence identity, at least 80% sequence identity, at least 85% sequence identity, at least 90% sequence identity, at least 91%, sequence identity at least 92% sequence identity, at least 93% sequence identity, at least 94% sequence identity, at least 95% sequence identity, at least 96% sequence identity, at least 97% sequence identity, at least 98% sequence identity, or at least 99% sequence identity to CDRs of a heavy chain variable domain selected from any one of SEQ ID NO:1, SEQ ID NO: 2, SEQ ID NO: 3, or SEQ ID NO: 4.

In some embodiments, an anti-dsRNA antibody or antigen-binding polypeptide described herein comprises anti-dsRNA HCDR1, anti-dsRNA HCDR2, and an anti-dsRNA HCDR3 having about 70% sequence identity, about 75% sequence identity, about 80% sequence identity, about 85% sequence identity, about 90% sequence identity, about 91%, sequence identity about 92% sequence identity, about 93% sequence identity, about 94% sequence identity, about 95% sequence identity, about 96% sequence identity, about 97% sequence identity, about 98% sequence identity, or about 99% sequence identity to CDRs of a heavy chain variable domain selected from any one of SEQ ID NO:1, SEQ ID NO:2, SEQ ID NO: 3, or SEQ ID NO: 4.

In some embodiments, an anti-dsRNA antibody or antigen-binding polypeptide described herein comprises anti-dsRNA HCDR1, anti-dsRNA HCDR2, and an anti-dsRNA HCDR3 having 70% sequence identity, 75% sequence identity, 80% sequence identity, 85% sequence identity, 90% sequence identity, 91%, sequence identity 92% sequence identity, 93% sequence identity, 94% sequence identity, 95% sequence identity, 96% sequence identity, 97% sequence identity, 98% sequence identity, or 99% sequence identity to CDRs of a heavy chain variable domain selected from any one of SEQ ID NO:1, SEQ ID NO:2, SEQ ID NO: 3, or SEQ ID NO: 4.

As will be readily understood by one of ordinary skill in the art, any anti-dsRNA HCDR sequence described herein can be readily combined with any other antibody or antigen-binding polypeptide sequences or domains described herein or otherwise known in the art. Combining of such anti-dsRNA HCDRs with other antibody or antigen-binding polypeptide sequences or domains can be readily achieved using known and understood molecular biology techniques.

### B. Exemplary Heavy Chain Variable Domains (VHs)

The present disclosure provides, among other things, anti-dsRNA heavy chain variable domain sequences (VHs). In some embodiments, an anti-dsRNA antibody or antigen-binding polypeptide described herein comprises an anti-dsRNA heavy chain variable domain (VH).

Anti-dsRNA heavy chain variable domains of the present disclosure can comprise, one, two, or all of an anti-dsRNA HCDR1, an anti-dsRNA HCDR2, and/or an anti-dsRNA HCDR3. In some embodiments, an anti-dsRNA heavy chain variable domain comprises an anti-dsRNA HCDR1. In some embodiments, an anti-dsRNA heavy chain variable domain comprises an anti-dsRNA HCDR2. In some embodiments, an anti-dsRNA heavy chain variable domain comprises an anti-dsRNA HCDR3. In some embodiments, an anti-dsRNA heavy chain variable domain comprises an anti-dsRNA HCDR1, an anti-dsRNA HCDR2, and an anti-dsRNA HCDR3 described herein.

In some embodiments, an anti-dsRNA antibody or antigen-binding polypeptide described herein comprises an anti-dsRNA heavy chain variable domain of SEQ ID NO: 1, SEQ ID NO: 2, SEQ ID NO: 3, and/or SEQ ID NO: 4.

In some embodiments, an anti-dsRNA antibody or antigen-binding polypeptide described herein comprises an anti-dsRNA heavy chain variable domain having at least 70% sequence identity, at least 75% sequence identity, at least 80% sequence identity, at least 85% sequence identity, at least 90% sequence identity, at least 91%, sequence identity at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO:1, SEQ ID NO: 2, SEQ ID NO: 3, or SEQ ID NO: 4.

In some embodiments, an anti-dsRNA antibody or antigen-binding polypeptide described herein comprises an anti-dsRNA heavy chain variable domain having about 70% sequence identity, about 75% sequence identity, about 80% sequence identity, about 85% sequence identity, about 90% sequence identity, about 91%, sequence identity about 92% sequence identity, about 93% sequence identity, about 94% sequence identity, about 95% sequence identity, about 96% sequence identity, about 97% sequence identity, about 98% sequence identity, or about 99% sequence identity to any one of SEQ ID NO:1, SEQ ID NO: 2, SEQ ID NO: 3, or SEQ ID NO: 4.

In some embodiments, an anti-dsRNA antibody or antigen-binding polypeptide described herein comprises an anti-dsRNA heavy chain variable domain having 70% sequence identity, 75% sequence identity, 80% sequence identity, 85% sequence identity, 90% sequence identity, 91%, sequence identity 92% sequence identity, 93% sequence identity, 94% sequence identity, 95% sequence identity, 96% sequence identity, 97% sequence identity, 98% sequence identity, or 99% sequence identity to any one of SEQ ID NO: 1, SEQ ID NO:2, SEQ ID NO: 3, or SEQ ID NO: 4.

Exemplary heavy chain variable domain amino acid sequences of the present disclosure are summarized in Table 1.

TABLE 1
Exemplary Heavy Chain Variable Domain amino acid sequences

| SEQ | | |
|---|---|---|
| ID NO: | Name | Amino acid sequence |
| 1 | Ab1 VH | EVQLQQSGPELVKPGASVKMSCKASGYTFANHVMHWVK |
| | | QKPGQGLEWIGYIYPYNDGTKYNEKFKGKATLTSDKSSST |
| | | AYMELSSLASEDSAVYYCARNNPAWFAYWGQGTLVTVS |
| | | A |
| 2 | Ab2 VH | EVQLQQSGPELVKPGASVKMSCKASGYTFANHVMHWVK |
| | | QKPGQGLEWIGYIYPYNDGTKYNEKFKGKATLTSDKSSST |
| | | AYMELSSLASEDSAVYYCARNNPAWFAYWGQGTLVTVS |
| | | A |
| 3 | Ab3 VH | EVQLQQSGPELVKPGASVKMSCKASGYTFANHVMHWVK |
| | | QKPGQGLEWIGYIYPYNDGTKYNEKFKGKATLTSDKSSST |
| | | AYMELSSLASEDSAVYYCASNYYFDYWGQGTTLTVSS |
| 4 | Ab4 VH | EVQLQQSGPELVKPGASVKMSCKASGYTFANHVMHWVK |
| | | QKPGQGLEWIGYIYPYNDGTKYNEKFKGKATLTSDKSSST |
| | | AYMELSSLASEDSAVYYCASNYYFDYWGQGTTLTVSS |

### C. Exemplary Heavy Chains (HCs)

The present disclosure provides, among other things, anti-dsRNA heavy chain sequences (HCs). In some embodiments, an anti-dsRNA antibody or antigen-binding polypeptide described herein comprises an anti-dsRNA heavy chain.

Anti-dsRNA heavy chains of the present disclosure can include any anti-dsRNA heavy chain variable domain described herein.

Anti-dsRNA heavy chains of the present disclosure can include any heavy chain constant domain disclosed herein or known in the art. In some embodiments, an anti-dsRNA heavy chain constant domain is of any class or subclass. In some embodiments, a heavy chain constant domain comprises, for example and without limitation, an amino acid sequence of one or more of an IgA, IgD, IgE, IgG, or IgM, including subclasses, which include, for example and without limitation, IgA1, IgA2, IgG1, IgG2, IgG3, IgG4. In some embodiments, a constant domain comprises a combination of more than one (e.g., at least 2, at least 3, at least 4) classes or subclasses of immunoglobulin heavy chain constant domain.

In some embodiments, an anti-dsRNA antibody or antigen-binding polypeptide described herein comprises one or more heavy chain constant domains of SEQ ID NO: 13-24.

In some embodiments, an anti-dsRNA antibody or antigen-binding polypeptide described herein comprises a heavy chain constant domain comprising a CH1, CH2, and/or CH3 having at least 70% sequence identity, at least 75% sequence identity, at least 80% sequence identity, at least 85% sequence identity, at least 90% sequence identity, at least 91%, sequence identity at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 13-24.

In some embodiments, an anti-dsRNA antibody or antigen-binding polypeptide described herein comprises a heavy chain constant domain comprising a CH1, CH2, and/or CH3 having about 70% sequence identity, about 75% sequence identity, about 80% sequence identity, about 85% sequence identity, about 90% sequence identity, about 91%, sequence identity about 92% sequence identity, about 93% sequence identity, about 94% sequence identity, about 95% sequence identity, about 96% sequence identity, about 97% sequence identity, about 98% sequence identity, or about 99% sequence identity to any one of SEQ ID NOs: 13-24.

In some embodiments, an anti-dsRNA antibody or antigen-binding polypeptide described herein comprises a heavy chain constant domain comprising a CH1, CH2, and/or CH3 having 70% sequence identity, 75% sequence identity, 80% sequence identity, 85% sequence identity, 90% sequence identity, 91%, sequence identity 92% sequence identity, 93% sequence identity, 94% sequence identity, 95% sequence identity, 96% sequence identity, 97% sequence identity, 98% sequence identity, or 99% sequence identity to any one of SEQ ID NOs: 13-24.

Exemplary heavy chain constant domains of the present disclosure are summarized in Table 2.

TABLE 2
Exemplary heavy chain constant domain amino acid sequences

| SEQ | | |
|---|---|---|
| ID NO: | Name | Amino acid sequence |
| 13 | Ab1 HC | AKTTAPSVYPLAPVCGDTTGSSVTLGCLVKGYFPEPVTLTW |
| | CH1 | NSGSLSSGVHTFPAVLQSDLYTLSSSVTVTSSTWPSQSITCNV |
| | | AHPASSTKVDKKI |
| 14 | Ab1 HC | EPRGPTIKPCPPCKCPAPNLLGGPSVFIFPPKIKDVLMISLSPIV |
| | hinge- | TCVVVDVSEDDPDVQISWFVNNVEVHTAQTQTHREDYNST |
| | CH2 | LRVVSALPIQHQDWMSGKEFKCKVNNKDLPAPIERTISKPK |
| 15 | Ab1 HC | GSVRAPQVYVLPPPEEEMTKKQVTLTCMVTDFMPEDIYVE |
| | CH3 | WTNNGKTELNYKNTEPVLDSDGSYFMYSKLRVEKKNWVE |
| | | RNSYSCSVVHEGLHNHHTTKSFSRTPGK |
| 16 | Ab2 HC | AKTTAPSVYPLAPVCGDTTGSSVTLGCLVKGYFPEPVTLTW |
| | CH1 | NSGSLSSGVHTFPAVLQSDLYTLSSSVTVTSSTWPSQSITCNV |
| | | AHPASSTKVDKKI |
| 17 | Ab2 HC | EPKSCDKTHTCPPCPAPELLGGPSVFLFPPKPKDTLMISRTPE |
| | hinge- | VTCVVVDVSHEDPEVKFNWYVDGVEVHNAKTKPREEQYN |
| | CH2 | STYRVVSVLTVLHQDWLNGKEYKCKVSNKALPAPIEKTISK |
| | | AK |
| 18 | Ab2 HC | GQPREPQVYTLPPSRDELTKNQVSLTCLVKGFYPSDIAVEWE |
| | CH3 | SNGQPENNYKTTPPVLDSDGSFFLYSKLTVDKSRWQQGNVF |
| | | SCSVMHEALHNHYTQKSLSLSPGK |
| 19 | Ab3 HC | AKTTAPSVYPLAPVCGDTTGSSVTLGCLVKGYFPEPVTLTW |
| | CH1 | NSGSLSSGVHTFPAVLQSDLYTLSSSVTVTSSTWPSQSITCNV |
| | | AHPASSTKVDKKI |
| 20 | Ab3 HC | EPRGPTIKPCPPCKCPAPNLLGGPSVFIFPPKIKDVLMISLSPIV |
| | hinge- | TCVVVDVSEDDPDVQISWFVNNVEVHTAQTQTHREDYNST |
| | CH2 | LRVVSALPIQHQDWMSGKEFKCKVNNKDLPAPIERTISKPK |
| 21 | Ab3 HC | GSVRAPQVYVLPPPEEEMTKKQVTLTCMVTDFMPEDIYVE |
| | CH3 | WTNNGKTELNYKNTEPVLDSDGSYFMYSKLRVEKKNWVE |
| | | RNSYSCSVVHEGLHNHHTTKSFSRTPGK |
| 22 | Ab4 HC | AKTTAPSVYPLAPVCGDTTGSSVTLGCLVKGYFPEPVTLTW |
| | CH1 | NSGSLSSGVHTFPAVLQSDLYTLSSSVTVTSSTWPSQSITCNV |
| | | AHPASSTKVDKKI |
| 23 | Ab4 HC | EPKSCDKTHTCPPCPAPELLGGPSVFLFPPKPKDTLMISRTPE |
| | hinge- | VTCVVVDVSHEDPEVKFNWYVDGVEVHNAKTKPREEQYN |
| | CH2 | STYRVVSVLTVLHQDWLNGKEYKCKVSNKALPAPIEKTISK |
| | | AK |
| 24 | Ab4 HC | GQPREPQVYTLPPSRDELTKNQVSLTCLVKGFYPSDIAVEWE |
| | CH3 | SNGQPENNYKTTPPVLDSDGSFFLYSKLTVDKSRWQQGNVF |
| | | SCSVMHEALHNHYTQKSLSLSPGK |

In some embodiments, an anti-dsRNA antibody or antigen-binding polypeptide described herein comprises an anti-dsRNA heavy chain of SEQ ID NO: 5, SEQ ID NO: 6, SEQ ID NO: 7, or SEQ ID NO: 8.

In some embodiments, an anti-dsRNA antibody or antigen-binding polypeptide described herein comprises a heavy chain sequence having at least 70% sequence identity, at least 75% sequence identity, at least 80% sequence identity, at least 85% sequence identity, at least 90% sequence identity, at least 91%, sequence identity at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 5-8.

In some embodiments, an anti-dsRNA antibody or antigen-binding polypeptide described herein comprises a heavy chain sequence having about 70% sequence identity, about 75% sequence identity, about 80% sequence identity, about 85% sequence identity, about 90% sequence identity, about 91%, sequence identity about 92% sequence identity, about 93% sequence identity, about 94% sequence identity, about 95% sequence identity, about 96% sequence identity, about 97% sequence identity, about 98% sequence identity, or about 99% sequence identity to any one of SEQ ID NOs: 5-8.

In some embodiments, an anti-dsRNA antibody or antigen-binding polypeptide described herein comprises a heavy chain sequence having 70% sequence identity, 75% sequence identity, 80% sequence identity, 85% sequence identity, 90% sequence identity, 91%, sequence identity 92% sequence identity, 93% sequence identity, 94% sequence identity, 95% sequence identity, 96% sequence identity, 97% sequence identity, 98% sequence identity, or 99% sequence identity to any one of SEQ ID NOs: 5-8.

Exemplary heavy chain amino acid sequences of the present disclosure are summarized in Table 3.

TABLE 3
Exemplary Heavy Chain amino acid sequences.

| SEQ | | |
|---|---|---|
| ID NO: | Name | Amino acid sequence |
| 5 | Ab1 HC | EVQLQQSGPELVKPGASVKMSCKASGYTFANHVMHWVKQ |
| | | KPGQGLEWIGYIYPYNDGTKYNEKFKGKATLTSDKSSSTAY |
| | | MELSSLASEDSAVYYCARNNPAWFAYWGQGTLVTVSAAK |
| | | TTAPSVYPLAPVCGDTTGSSVTLGCLVKGYFPEPVTLTWNS |
| | | GSLSSGVHTFPAVLQSDLYTLSSSVTVTSSTWPSQSITCNVA |
| | | HPASSTKVDKKIEPRGPTIKPCPPCKCPAPNLLGGPSVFIFPPK |
| | | IKDVLMISLSPIVTCVVVDVSEDDPDVQISWFVNNVEVHTAQ |
| | | TQTHREDYNSTLRVVSALPIQHQDWMSGKEFKCKVNNKDL |
| | | PAPIERTISKPKGSVRAPQVYVLPPPEEEMTKKQVTLTCMVT |
| | | DFMPEDIYVEWTNNGKTELNYKNTEPVLDSDGSYFMYSKL |
| | | RVEKKNWVERNSYSCSVVHEGLHNHHTTKSFSRTPGK |
| 6 | Ab2 HC | EVQLQQSGPELVKPGASVKMSCKASGYTFANHVMHWVKQ |
| | | KPGQGLEWIGYIYPYNDGTKYNEKFKGKATLTSDKSSSTAY |
| | | MELSSLASEDSAVYYCARNNPAWFAYWGQGTLVTVSAAK |
| | | TTAPSVYPLAPVCGDTTGSSVTLGCLVKGYFPEPVTLTWNS |
| | | GSLSSGVHTFPAVLQSDLYTLSSSVTVTSSTWPSQSITCNVA |
| | | HPASSTKVDKKIEPKSCDKTHTCPPCPAPELLGGPSVFLFPPK |
| | | PKDTLMISRTPEVTCVVVDVSHEDPEVKFNWYVDGVEVHN |
| | | AKTKPREEQYNSTYRVVSVLTVLHQDWLNGKEYKCKVSNK |
| | | ALPAPIEKTISKAKGQPREPQVYTLPPSRDELTKNQVSLTCLV |
| | | KGFYPSDIAVEWESNGQPENNYKTTPPVLDSDGSFFLYSKLT |
| | | VDKSRWQQGNVFSCSVMHEALHNHYTQKSLSLSPGK |
| 7 | Ab3 HC | EVQLQQSGPELVKPGASVKMSCKASGYTFANHVMHWVKQ |
| | | KPGQGLEWIGYIYPYNDGTKYNEKFKGKATLTSDKSSSTAY |
| | | MELSSLASEDSAVYYCASNYYFDYWGQGTTLTVSSAKTTA |
| | | PSVYPLAPVCGDTTGSSVTLGCLVKGYFPEPVTLTWNSGSLS |
| | | SGVHTFPAVLQSDLYTLSSSVTVTSSTWPSQSITCNVAHPAS |
| | | STKVDKKIEPRGPTIKPCPPCKCPAPNLLGGPSVFIFPPKIKDV |
| | | LMISLSPIVTCVVVDVSEDDPDVQISWFVNNVEVHTAQTQT |
| | | HREDYNSTLRVVSALPIQHQDWMSGKEFKCKVNNKDLPAPI |
| | | ERTISKPKGSVRAPQVYVLPPPEEEMTKKQVTLTCMVTDFM |
| | | PEDIYVEWTNNGKTELNYKNTEPVLDSDGSYFMYSKLRVE |
| | | KKNWVERNSYSCSVVHEGLHNHHTTKSFSRTPGK |
| 8 | Ab4 HC | EVQLQQSGPELVKPGASVKMSCKASGYTFANHVMHWVKQ |
| | | KPGQGLEWIGYIYPYNDGTKYNEKFKGKATLTSDKSSSTAY |
| | | MELSSLASEDSAVYYCASNYYFDYWGQGTTLTVSSAKTTA |
| | | PSVYPLAPVCGDTTGSSVTLGCLVKGYFPEPVTLTWNSGSLS |
| | | SGVHTFPAVLQSDLYTLSSSVTVTSSTWPSQSITCNVAHPAS |
| | | STKVDKKIEPKSCDKTHTCPPCPAPELLGGPSVFLFPPKPKDT |
| | | LMISRTPEVTCVVVDVSHEDPEVKFNWYVDGVEVHNAKTK |
| | | PREEQYNSTYRVVSVLTVLHQDWLNGKEYKCKVSNKALPA |
| | | PIEKTISKAKGQPREPQVYTLPPSRDELTKNQVSLTCLVKGF |
| | | YPSDIAVEWESNGQPENNYKTTPPVLDSDGSFFLYSKLTVD |
| | | KSRWQQGNVFSCSVMHEALHNHYTQKSLSLSPGK |

### D. Exemplary Light Chain Complementary Determining Regions (LCDRs)

The present disclosure, among other things, provides anti-dsRNA LCDR sequences. In some embodiments, an anti-dsRNA antibody or antigen-binding polypeptide described herein comprises one or more of an anti-dsRNA LCDR1, an anti-dsRNA LCDR2, and/or an anti-dsRNA LCDR3.

In some embodiments, an anti-dsRNA antibody or antigen-binding polypeptide comprises CDRs of a light chain variable domain sequence SEQ ID NO: 9 or SEQ ID NO: 10.

In some embodiments, an anti-dsRNA antibody or antigen-binding polypeptide described herein comprises anti-dsRNA LCDR1, anti-dsRNA LCDR2, and an anti-dsRNA LCDR3 having at least 70% sequence identity, at least 75% sequence identity, at least 80% sequence identity, at least 85% sequence identity, at least 90% sequence identity, at least 91%, sequence identity at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 9 or SEQ ID NO: 10.

In some embodiments, an anti-dsRNA antibody or antigen-binding polypeptide described herein comprises anti-dsRNA LCDR1, anti-dsRNA LCDR2, and an anti-dsRNA LCDR3 having about 70% sequence identity, about 75% sequence identity, about 80% sequence identity, about 85% sequence identity, about 90% sequence identity, about 91%, sequence identity about 92% sequence identity, about 93% sequence identity, about 94% sequence identity, about 95% sequence identity, about 96% sequence identity, about 97% sequence identity, about 98% sequence identity, or about 99% sequence identity to any one of SEQ ID NO: 9 or SEQ ID NO: 10.

In some embodiments, an anti-dsRNA antibody or antigen-binding polypeptide described herein comprises anti-dsRNA LCDR1, anti-dsRNA LCDR2, and an anti-dsRNA LCDR3 having 70% sequence identity, 75% sequence identity, 80% sequence identity, 85% sequence identity, 90% sequence identity, 91%, sequence identity 92% sequence identity, 93% sequence identity, 94% sequence identity, 95% sequence identity, 96% sequence identity, 97% sequence identity, 98% sequence identity, or 99% sequence identity to any one of SEQ ID NO: 9 or SEQ ID NO: 10.

As will be readily understood by one of ordinary skill in the art, any anti-dsRNA LCDR sequence described herein can be readily combined with any other antibody or antigen-binding polypeptide sequences or domains described herein or otherwise known in the art. Combining of such anti-dsRNA LCDRs with other antibody or antigen-binding polypeptide sequences or domains can be readily achieved using known and understood molecular biology techniques.

### E. Exemplary Light Chain Variable Domains (VLs)

The present disclosure provides, among other things, anti-dsRNA light chain variable domain sequences (VLs). In some embodiments, an anti-dsRNA antibody or antigen-binding polypeptide described herein comprises an anti-dsRNA light chain variable domain (VL).

Anti-dsRNA light chain variable domains of the present disclosure can comprise, one, two, or all of an anti-dsRNA LCDR1, an anti-dsRNA LCDR2, and/or an anti-dsRNA LCDR3. In some embodiments, an anti-dsRNA light chain variable domain comprises an anti-dsRNA LCDR1. In some embodiments, an anti-dsRNA light chain variable domain comprises an anti-dsRNA LCDR2. In some embodiments, an anti-dsRNA light chain variable domain comprises an anti-dsRNA LCDR3. In some embodiments, an anti-dsRNA light chain variable domain comprises an anti-dsRNA LCDR1, an anti-dsRNA LCDR2, and an anti-dsRNA LCDR3 described herein.

In some embodiments, an anti-dsRNA antibody or antigen-binding polypeptide described herein comprises an anti-dsRNA light chain variable domain of SEQ ID NO: 9 or SEQ ID NO: 10.

In some embodiments, an anti-dsRNA antibody or antigen-binding polypeptide described herein comprises an anti-dsRNA light chain variable domain having at least 70% sequence identity, at least 75% sequence identity, at least 80% sequence identity, at least 85% sequence identity, at least 90% sequence identity, at least 91%, sequence identity at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 9 or SEQ ID NO: 10.

In some embodiments, an anti-dsRNA antibody or antigen-binding polypeptide described herein comprises an anti-dsRNA light chain variable domain having about 70% sequence identity, about 75% sequence identity, about 80% sequence identity, about 85% sequence identity, about 90% sequence identity, about 91%, sequence identity about 92% sequence identity, about 93% sequence identity, about 94% sequence identity, about 95% sequence identity, about 96% sequence identity, about 97% sequence identity, about 98% sequence identity, or about 99% sequence identity to any one of SEQ ID NO: 9 or SEQ ID NO: 10.

In some embodiments, an anti-dsRNA antibody or antigen-binding polypeptide described herein comprises an anti-dsRNA light chain variable domain having 70% sequence identity, 75% sequence identity, 80% sequence identity, 85% sequence identity, 90% sequence identity, 91%, sequence identity 92% sequence identity, 93% sequence identity, 94% sequence identity, 95% sequence identity, 96% sequence identity, 97% sequence identity, 98% sequence identity, or 99% sequence identity to any one of SEQ ID NO: 9 or SEQ ID NO: 10.

Exemplary light chain variable domain amino acid sequences of the present disclosure are summarized in Table 4.

TABLE 4
Exemplary Light Chain Variable Domain amino acid sequences.

| SEQ | | |
|---|---|---|
| ID NO: | Name | Amino acid sequence |
| 9 | Ab1 LC | NIMMTQSPSSLAVSAGEKVTMSCKSSQSVLYSSNQKNYLA |
| | | WYQQKPGQSPKLLIYWASTRESGVPDRFTGSGSGTDFTLTIS |
| | | SVQAEDLAVYYCHQYLSSYTFGGGTKLEIKR |
| 10 | Ab3 LC | QIVLTQSPAIMSASPGEKVTMTCSASSSVSYMHWYQQKSGT |
| | | SPKRWIYDTSKLASGVPARFSGSGSGTSYSLTISSMEAEDAA |
| | | TYYCQQWSSNPYTFGGGTKLEIKR |

### F. Exemplary Anti-dsRNA Light Chains (LCs)

The present disclosure provides, among other things, anti-dsRNA light chain sequences (LCs). In some embodiments, an anti-dsRNA antibody or antigen-binding polypeptide described herein comprises an anti-dsRNA light chain.

Anti-dsRNA light chains of the present disclosure can include any anti-dsRNA light chain variable domain described herein.

Anti-dsRNA light chains of the present disclosure can include any light chain constant domain disclosed herein or known in the art. In some embodiments, an anti-dsRNA antibody or antigen-binding polypeptide comprises an anti-dsRNA light chain that includes any light chain constant domain, e.g., a light chain constant domain readily known and understood in the art. In some embodiments, a light chain constant domain is or comprises a kappa light chain constant domain. In some embodiments, a light chain constant domain is or comprises a lambda light chain constant domain.

In some embodiments, an anti-dsRNA antibody or antigen-binding polypeptide described herein comprises one or more light chain constant domains of SEQ ID NO: 25 or SEQ ID NO: 26.

In some embodiments, an anti-dsRNA antibody or antigen-binding polypeptide described herein comprises a light chain constant domain having at least 70% sequence identity, at least 75% sequence identity, at least 80% sequence identity, at least 85% sequence identity, at least 90% sequence identity, at least 91%, sequence identity at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 25 or SEQ ID NO: 26.

In some embodiments, an anti-dsRNA antibody or antigen-binding polypeptide described herein comprises a light chain constant domain having about 70% sequence identity, about 75% sequence identity, about 80% sequence identity, about 85% sequence identity, about 90% sequence identity, about 91%, sequence identity about 92% sequence identity, about 93% sequence identity, about 94% sequence identity, about 95% sequence identity, about 96% sequence identity, about 97% sequence identity, about 98% sequence identity, or about 99% sequence identity to any one of SEQ ID NO: 25 or SEQ ID NO: 26.

In some embodiments, an anti-dsRNA antibody or antigen-binding polypeptide described herein comprises a light chain constant domain having 70% sequence identity, 75% sequence identity, 80% sequence identity, 85% sequence identity, 90% sequence identity, 91%, sequence identity 92% sequence identity, 93% sequence identity, 94% sequence identity, 95% sequence identity, 96% sequence identity, 97% sequence identity, 98% sequence identity, or 99% sequence identity to any one of SEQ ID NO: 25 or SEQ ID NO: 26.

Exemplary light chain constant domains of the present disclosure are summarized in Table 5.

TABLE 5
Exemplary light chain constant domain (CL) amino acid sequences.

| SEQ | | |
|---|---|---|
| ID NO: | Name | Amino acid sequence |
| 25 | Ab1 LC | ADAAPTVSIFPPSSEQLTSGGASVVCFLNNFYPKDINVKWKI |
| | CL | DGSERQNGVLNSWTDQDSKDSTYSMSSTLTLTKDEYERHN |
| | | SYTCEATHATSTSPIVKSFNRNEC |
| 26 | Ab3 LC | ADAAPTVSIFPPSSEQLTSGGASVVCFLNNFYPKDINVKWKI |
| | CL | DGSERQNGVLNSWTDQDSKDSTYSMSSTLTLTKDEYERHN |
| | | SYTCEATHKTSTSPIVKSFNRNEC |

In some embodiments, an anti-dsRNA antibody or antigen-binding polypeptide described herein comprises an anti-dsRNA light chain of SEQ ID NO: 11 or SEQ ID NO: 12.

In some embodiments, an anti-dsRNA antibody or antigen-binding polypeptide described herein comprises a light chain sequence having at least 70% sequence identity, at least 75% sequence identity, at least 80% sequence identity, at least 85% sequence identity, at least 90% sequence identity, at least 91%, sequence identity at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NO: 11 or SEQ ID NO: 12.

In some embodiments, an anti-dsRNA antibody or antigen-binding polypeptide described herein comprises a light chain sequence having about 70% sequence identity, about 75% sequence identity, about 80% sequence identity, about 85% sequence identity, about 90% sequence identity, about 91%, sequence identity about 92% sequence identity, about 93% sequence identity, about 94% sequence identity, about 95% sequence identity, about 96% sequence identity, about 97% sequence identity, about 98% sequence identity, or about 99% sequence identity to any one of SEQ ID NO: 11 or SEQ ID NO: 12.

In some embodiments, an anti-dsRNA antibody or antigen-binding polypeptide described herein comprises a light chain sequence having 70% sequence identity, 75% sequence identity, 80% sequence identity, 85% sequence identity, 90% sequence identity, 91%, sequence identity 92% sequence identity, 93% sequence identity, 94% sequence identity, 95% sequence identity, 96% sequence identity, 97% sequence identity, 98% sequence identity, or 99% sequence identity to any one of SEQ ID NO: 11 or SEQ ID NO: 12.

Exemplary light chain amino acid sequences of the present disclosure are summarized in Table 6.

TABLE 6
Exemplary Light Chain amino acid sequences.

| SEQ | | |
|---|---|---|
| ID NO: | Name | Amino acid sequence |
| 11 | Ab1 LC | NIMMTQSPSSLAVSAGEKVTMSCKSSQSVLYSSNQKNYLA |
| | | WYQQKPGQSPKLLIYWASTRESGVPDRFTGSGSGTDFTLTIS |
| | | SVQAEDLAVYYCHQYLSSYTFGGGTKLEIKRADAAPTVSIFP |
| | | PSSEQLTSGGASVVCFLNNFYPKDINVKWKIDGSERQNGVL |
| | | NSWTDQDSKDSTYSMSSTLTLTKDEYERHNSYTCEATHATS |
| | | TSPIVKSFNRNEC |
| 12 | Ab3 LC | QIVLTQSPAIMSASPGEKVTMTCSASSSVSYMHWYQQKSGT |
| | | SPKRWIYDTSKLASGVPARFSGSGSGTSYSLTISSMEAEDAA |
| | | TYYCQQWSSNPYTFGGGTKLEIKRADAAPTVSIFPPSSEQLT |
| | | SGGASVVCFLNNFYPKDINVKWKIDGSERQNGVLNSWTDQ |
| | | DSKDSTYSMSSTLTLTKDEYERHNSYTCEATHKTSTSPIVKS |
| | | FNRNEC |

### G. Specific Embodiments of Disclosed Antibodies

In some embodiments, an anti-dsRNA antibody or antigen-binding polypeptide of the present disclosure comprises a heavy chain comprising CDRs of a heavy chain variable domain sequence SEQ ID NO: 1, SEQ ID NO: 2, SEQ ID NO: 3, or SEQ ID NO: 4 and a light chain comprising CDRs of a light chain variable domain sequence SEQ ID NO: 9 or SEQ ID NO: 10.

In some embodiments, an anti-dsRNA antibody or antigen-binding polypeptide of the present disclosure comprises a heavy chain variable domain sequence that comprises an amino acid sequence selected from the group consisting of SEQ ID NO: 1, SEQ ID NO: 2, SEQ ID NO: 3, and SEQ ID NO: 4. In some embodiments, an anti-dsRNA antibody or antigen-binding polypeptide of the present disclosure comprises a light chain variable domain sequence that comprises an amino acid sequence selected from the group consisting of SEQ ID NO: 9 and SEQ ID NO: 10.

In some embodiments, an anti-dsRNA antibody or antigen-binding polypeptide of the present disclosure comprises a heavy chain that comprises an amino acid sequence selected from the group consisting of SEQ ID NO: 5, SEQ ID NO:6, SEQ ID NO: 7, and SEQ ID NO: 8. In some embodiments, an anti-dsRNA antibody or antigen-binding polypeptide of the present disclosure comprises a light chain that comprises an amino acid sequence selected from the group consisting of SEQ ID NO: 11 and SEQ ID NO: 12.

In some embodiments, an anti-dsRNA antibody of the present disclosure comprises two light chains of SEQ ID NO: 11 and two heavy chains of SEQ ID NO: 5 (hereafter referred to as “Antibody 1 (Ab1)”). In some embodiments, an anti-dsRNA antibody of the present disclosure comprises two light chains of SEQ ID NO: 11 and two heavy chains of SEQ ID NO: 6 (hereafter referred to as “Antibody 2 (Ab2)”). In some embodiments, an anti-dsRNA antibody of the present disclosure comprises two light chains of SEQ ID NO: 12 and two heavy chains of SEQ ID NO: 7 (hereafter referred to as “Antibody 3 (Ab3)”). In some embodiments, an anti-dsRNA antibody of the present disclosure comprises two light chains of SEQ ID NO: 12 and two heavy chains of SEQ ID NO: 8 (hereafter referred to as “Antibody 4 (Ab4)”).

### H. Epitopes and Binding

An anti-dsRNA antibody or antigen-binding polypeptide of the present disclosure can specifically bind to dsRNA in a sequence-independent manner and independent of the length of the dsRNA. Without wishing to be bound by any one theory, it is believed that antibodies and antigen-binding polypeptides of the present disclosure specifically bind to dsRNA by way of its unique electrostatic characteristics, particularly the phosphate backbone of the dsRNA molecule.

In some embodiments, disclosed anti-dsRNA antibodies and antigen-binding polypeptides may be defined by sequence, or by functional characteristics. For instance, disclosed antibodies and antigen-binding polypeptides, can have a K_{D} of at least 3.0×10⁻⁸, at least 2.5×10⁻⁸, at least 2.0×10⁻⁸, at least 1.5×10⁻⁸, at least 1.0×10⁻⁸, at least 0.5×10⁻⁸, at least 9.95×10⁻⁹, at least 9.90×10⁻⁹, at least 9.85×10⁻⁹, at least 9.80×10⁻⁹, at least 9.75×10⁻⁹, at least 9.70×10⁻⁹, at least 9.65×10⁻⁹, at least 9.60×10⁻⁹, at least 9.55×10⁻⁹, at least 9.5×10⁻⁹, at least 9.45×10⁻⁹, at least 9.40×10⁻⁹, at least 9.35×10⁻⁹, at least 9.30×10⁻⁹, at least 9.25×10⁻⁹, at least 9.20×10⁻⁹, at least 9.15×10⁻⁹, at least 9.10×10⁻⁹, at least 9.05×10⁻⁹, at least 9.0×10⁻⁹, at least 8.95×10⁻⁹, at least 8.90×10⁻⁹, at least 8.85×10⁻⁹, at least 8.80×10⁻⁹, at least 8.75×10⁻⁹, at least 8.70×10⁻⁹, at least 8.65×10⁻⁹, at least 8.60×10⁻⁹, at least 8.55×10⁻⁹, at least 8.5×10⁻⁹, at least 8.45×10⁻⁹, at least 8.40×10⁻⁹, at least 8.35×10⁻⁹, at least 8.30×10⁻⁹, at least 8.25×10⁻⁹, at least 8.20×10⁻⁹, at least 8.15×10⁻⁹, at least 8.10×10⁻⁹, at least 8.05×10⁻⁹, at least 8.0×10⁻⁹, at least 7.95×10⁻⁹, at least 7.90×10⁻⁹, at least 7.85×10⁻⁹, at least 7.80×10⁻⁹, at least 7.75×10⁻⁹, at least 7.70×10⁻⁹, at least 7.65×10⁻⁹, at least 7.60×10⁻⁹, at least 7.55×10⁻⁹, at least 7.5×10⁻⁹, at least 7.45×10⁻⁹, at least 7.40×10⁻⁹, at least 7.35×10⁻⁹, at least 7.30×10⁻⁹, at least 7.25×10⁻⁹, at least 7.20×10⁻⁹, at least 7.15×10⁻⁹, at least 7.10×10⁻⁹, at least 7.05×10⁻⁹, at least 7.0×10⁻⁹, at least 6.95×10⁻⁹, at least 6.90×10⁻⁹, at least 6.85×10⁻⁹, at least 6.80×10⁻⁹, at least 6.75×10⁻⁹, at least 6.70×10⁻⁹, at least 6.65×10⁻⁹, at least 6.60×10⁻⁹, at least 6.55×10⁻⁹, at least 6.5×10⁻⁹, at least 6.45×10⁻⁹, at least 6.40×10⁻⁹, at least 6.35×10⁻⁹, at least 6.30×10⁻⁹, at least 6.25×10⁻⁹, at least 6.20×10⁻⁹, at least 6.15×10⁻⁹, at least 6.10×10⁻⁹, at least 6.05×10⁻⁹, at least 6.0×10⁻⁹, at least 5.95×10⁻⁹, at least 5.90×10⁻⁹, at least 5.85×10⁻⁹, at least 5.80×10⁻⁹, at least 5.75×10⁻⁹, at least 5.70×10⁻⁹, at least 5.65×10⁻⁹, at least 5.60×10⁻⁹, at least 5.55×10⁻⁹, at least 5.5×10⁻⁹, at least 5.45×10⁻⁹, at least 5.40×10⁻⁹, at least 5.35×10⁻⁹, at least 5.30×10⁻⁹, at least 5.25×10⁻⁹, at least 5.20×10⁻⁹, at least 5.15×10⁻⁹, at least 5.10×10⁻⁹, at least 5.05×10⁻⁹, at least 5.0×10⁻⁹, at least 4.95×10⁻⁹, at least 4.90×10⁻⁹, at least 4.85×10⁻⁹, at least 4.80×10⁻⁹, at least 4.75×10⁻⁹, at least 4.70×10⁻⁹, at least 4.65×10⁻⁹, at least 4.60×10⁻⁹, at least 4.55×10⁻⁹, at least 4.5×10⁻⁹, at least 4.45×10⁻⁹, at least 4.40×10⁻⁹, at least 4.35×10⁻⁹, at least 4.30×10⁻⁹, at least 4.25×10⁻⁹, at least 4.20×10⁻⁹, at least 4.15×10⁻⁹, at least 4.10×10⁻⁹, at least 4.05×10⁻⁹, at least 4.0×10⁻⁹, at least 3.95×10⁻⁹, at least 3.90×10⁻⁹, at least 3.85×10⁻⁹, at least 3.80×10⁻⁹, at least 3.75×10⁻⁹, at least 3.70×10⁻⁹, at least 3.65×10⁻⁹, at least 3.60×10⁻⁹, at least 3.55×10⁻⁹, at least 3.5×10⁻⁹, at least 3.45×10⁻⁹, at least 3.40×10⁻⁹, at least 3.35×10⁻⁹, at least 3.30×10⁻⁹, at least 3.25×10⁻⁹, at least 3.20×10⁻⁹, at least 3.15×10⁻⁹, at least 3.10×10⁻⁹, at least 3.05×10⁻⁹, at least 3.0×10⁻⁹, at least 2.95×10⁻⁹, at least 2.90×10⁻⁹, at least 2.85×10⁻⁹, at least 2.80×10⁻⁹, at least 2.75×10⁻⁹, at least 2.70×10⁻⁹, at least 2.65×10⁻⁹, at least 2.60×10⁻⁹, at least 2.55×10⁻⁹, at least 2.5×10⁻⁹, at least 2.45×10⁻⁹, at least 2.40×10⁻⁹, at least 2.35×10⁻⁹, at least 2.30×10⁻⁹, at least 2.25×10⁻⁹, at least 2.20×10⁻⁹, at least 2.15×10⁻⁹, at least 2.10×10⁻⁹, at least 2.05×10⁻⁹, at least 2.0×10⁻⁹, at least 1.95×10⁻⁹, at least 1.90×10⁻⁹, at least 1.85×10⁻⁹, at least 1.80×10⁻⁹, at least 1.75×10⁻⁹, at least 1.70×10⁻⁹, at least 1.65×10⁻⁹, at least 1.60×10⁻⁹, at least 1.55×10⁻⁹, at least 1.5×10⁻⁹, at least 1.45×10⁻⁹, at least 1.40×10⁻⁹, at least 1.35×10⁻⁹, at least 1.30×10⁻⁹, at least 1.25×10⁻⁹, at least 1.20×10⁻⁹, at least 1.15×10⁻⁹, at least 1.10×10⁻⁹, at least 1.05×10⁻⁹, at least 1.0×10⁻⁹, at least 0.95×10⁻⁹, at least 0.90×10⁻⁹, at least 0.85×10⁻⁹, at least 0.80×10⁻⁹, at least 0.75×10⁻⁹, at least 0.70×10⁻⁹, at least 0.65×10⁻⁹, at least 0.60×10⁻⁹, at least 0.55×10⁻⁹, at least 0.5×10⁻⁹, at least 0.45×10⁻⁹, at least 0.40×10⁻⁹, at least 0.35×10⁻⁹, at least 0.30×10⁻⁹, at least 0.25×10⁻⁹, at least 0.20×10⁻⁹, at least 0.15×10⁻⁹, at least 0.10×10⁻⁹, at least 0.05×10⁻⁹, at least 9.5×10-10, at least 9.0×10-10, at least 8.5×10-10, at least 8.0×10-10, or any value in between. For example, the disclosed antibodies and antigen-binding polypeptides can have K_{D} values of 8.2×10⁻¹⁰, 2.31×10⁻⁹ 8.24×10⁻⁹, 3.25×10⁻⁹, 3.46×10⁻⁹, 1.91×10⁻⁹, 7.97×10⁻⁸, 2.41×10⁻⁸, 9.5×10⁻¹⁰, or 8.6×10⁻¹⁰.

In some embodiments, disclosed antibodies and antigen-binding polypeptides can have IC₅₀ values between 4.0×10⁻⁵ to 9.5×10⁻¹⁰ μg/mL of any value in between. For example, disclosed antibodies and antigen-binding polypeptides can have IC₅₀ values of 9.19×10⁻⁷, 4.156×10⁻⁵, 9.984×10⁻⁷, 1.037×10⁻⁶, or 3.653×10⁻⁶.

### I. Labels

In some embodiments of the present disclosure, an anti-dsRNA antibody or antigen-binding polypeptide is associated with a label (e.g., a detectable label). In some embodiments, an antibody or antigen-binding polypeptide as described herein can be covalently or non-covalently associated with a label. In some embodiments, an antibody or antigen-binding polypeptide as described herein can be directly or indirectly associated with a label. In some embodiments, an antibody or antigen-binding polypeptide as described herein can be associated with a label via a linker. In some embodiments, an antibody or antigen-binding polypeptide as described herein can be fused or conjugated with a label. In some embodiments, an antibody or antigen-binding polypeptide described herein is associated with a label via a polypeptide terminus of the antibody or antigen-binding polypeptide. In some embodiments, an antibody or antigen-binding polypeptide described herein is associated with a label via a non-terminal residue of the antibody or antigen-binding polypeptide. In some embodiments, an antibody or antigen-binding polypeptide as described herein is associated with a label via incorporation of the label into the molecular structure of the antibody or antigen-binding polypeptide. In some embodiments, an antibody or antigen-binding polypeptide described herein can be associated with a plurality of the same or different labels via any mechanism described herein or otherwise known in the art, which the same or different labels can be associated with the antibody or antigen-binding polypeptide via the same or different mechanisms of associate as disclosed herein or otherwise known in the art.

Labels of the present disclosure include, without limitation, a label that produces, is capable of producing, or is capable of contributing to production of a signal detectable by any means known in the art. In some embodiments, a label produces, is capable of producing, or is capable of contributing to production of a single detectable by, without limitation, visual means, spectroscopic means, photochemical means, biochemical means, immunochemical means, electromagnetic means, radiochemical means, chemical means, fluorescence, chemifluorescence, electrochemiluminescence, or chemiluminescence. In some embodiments, a label is a fluorescent label, radioactive label, paramagnetic label, chemiluminescent label, bioluminescent label, colorimetric label, polypeptide, enzyme, and/or ligand.

Labels include, for example and without limitation, green fluorescent protein (GFP), red fluorescent protein (RFP), rhodamine, rhodamine-derived labels, fluorescein, fluorescein-derived labels, naphthalene, naphthalene-derived labels, coumarin, coumarin-derived labels, phycobiliproteins, and derivatives such as phycoerythrin and phycocyanin, luciferase, beta-galactosidase, chromophores, phenolphthalein, malachite green, nitroaromatics such as nitrophenyl, diazo dyes, dabsyl (4-dimethylaminoazobenzene-4′-sulfonyl), His tag, and biotin-binding moieties such as streptavidin or avidin. In some embodiments, a label can be a radioisotope or radiolabel. In some embodiments, a label may be a small molecule, a fluorescent dye, or a compound that may be detected by x-rays or electromagnetic radiation. In some embodiments, a label can be a catalytic substrate of an enzyme, wherein activity of enzyme with substrate produces a detectable signal. Enzyme detectable moieties of the present invention include, without limitation, peroxidase, horseradish peroxidase (HRP), alkaline phosphatase (AP), glucose oxidase, β-galactosidase, acetylcholinesterase, or catalase. In some such embodiments, the choice of label can depend on the required assay sensitivity and/or instrumentation available for signal detection.

### Methods of Detection and Quantification

The present disclosure provides methods for detecting and quantifying RNA (e.g., dsRNA) that represent a significant improvement over currently conventional dot blot protocols. In particular, the disclosed methods utilize antibodies that can bind to RNA in a sequence-independent manner, which allows these antibodies to be used in an ELISA format for detecting RNA. ELISA methods routinely use a capture antibody, which is bound to a solid substrate, such as the well of a plate, to bind a target molecule (e.g., dsRNA) when a sample containing the target molecule is applied to the substrate. A detection antibody is applied thereafter (often after a washing step), and the detection antibody also binds to the target molecule. The detection antibody may either be directed labeled, for example, with a fluorophore, chemiluminescent agent, enzyme, or other detectable label, or the detection antibody may be subsequently bound by a further secondary antibody that is labeled and which binds to the detection antibody.

The presently disclosed methods are further unique in that the capture antibody and detection antibody may comprise the same CDR sequences or variable domain sequences. This is uncommon in ELISA assays, as most antibodies recognize a specific region or epitope of a target molecule, and therefore such antibodies would compete for binding the target molecule. For example, if an ELISA designed to detect a specific protein were to utilize capture and detection antibodies that bound the same epitope of the desired protein, then the detection antibody may be unable to bind and provide a positive signal (e.g., based on the presence of the label) if the epitope is already occupied by the capture antibody. As a result, using a capture antibody and a detection antibody that have the same CDRs or variable sequences is, under most circumstances, counterintuitive.

The present methods, however, are unique because the disclosed antibodies are not sequence-specific, and therefore both the capture antibody and the detection antibody are capable of binding a target RNA molecule at the same time. This improves the consistency of detection and the dynamic range of a given assay using such sequence-independent antibodies.

Depending on the format of the method and, more specifically, whether the detection antibody is directly labeled or indirectly labeled via a secondary antibody, it may be beneficial for the capture antibody and the detection antibody to comprise either (i) different Fc domains, or (ii) different constant regions. In particular, when using a secondary antibody to indirectly label the detection antibody, it is beneficial to utilize different Fc domains or constant regions between the capture antibody and detection antibody such that the secondary antibody does not bind to the capture antibody and product a false positive result. For example, even when the capture antibody and detection antibody comprise the same CDRs or variable domains (e.g., VH/VL), the capture antibody may comprise a mouse Fc domain (or constant region) while the detection antibody comprises a human Fc domain (or constant region). In this way, a secondary antibody can be chosen based on its specificity to, for example, a human Fc domain, thereby allowing the secondary antibody to bind the detection antibody and not the capture antibody. Further embodiments of such methods are discussed in more detail below.

In some embodiments, the present disclosure provides methods of detecting and/or quantifying dsRNA in a sample using an ELISA. Any ELISA known in the art can be used in accordance with technologies disclosed herein. Known ELISA methods include, for example, direct assay ELISAs, indirect assay ELISAs, and capture assay (“sandwich”) ELISAs (see, e.g., Gan, S. et al., J Invest Dermatol 133.9 (2013)).

In some embodiments, an ELISA comprises binding (e.g., immobilizing) a capture antibody to the surface of a plate (e.g., polystyrene plates, microtiter plates coated with the positively charged protamine sulfate). A capture antibody can be immobilized on the surface of a plate by direct adsorption to the plate. In some embodiments, a target of interest is immobilized on the surface of a plate via a capture antibody (e.g., an antibody or antigen-binding polypeptide that specifically binds a target of interest as described herein) that has been attached to the surface of the plate, thereby producing a first complex comprising the target of interest (e.g., dsRNA) and the capture antibody. All of the disclosed antibodies may be suitably used as capture antibodies.

In some embodiments, a blocking buffer may be added to the plates to cover any remaining available binding surfaces of the plates (e.g., polystyrene plates) and/or to reduce non-specific binding of a non-target of interest to the capture antibody (e.g., by adsorption, by non-specific binding to a capture-antibody).

In some embodiments, the disclosed methods then comprises applying a detection antibody (e.g., a second antibody that specifically binds the target of interest). In some embodiments, a detection antibody is or comprises an antibody or antigen-binding polypeptide of the present disclosure. In some embodiments, the capture antibody and the detection antibody comprise the same CDRs or variable domains (e.g., VH and VL domains described herein). In some embodiments, the capture antibody and the detection antibody are from separate species. In some embodiments, the capture antibody and the detection antibody comprise Fc domains from different species (e.g., mouse and human). In some embodiments, the capture antibody and the detection antibody comprise at least one constant region from a different species (e.g., mouse and human). In some embodiments, the capture antibody comprises a murine heavy chain constant region (i.e., CH1, CH2, and CH3) and the detection antibody comprises a human heavy chain constant region (i.e., CH1, CH2, and CH3). In some embodiments, the capture antibody comprises a human heavy chain constant region (i.e., CH1, CH2, and CH3) and the detection antibody comprises a murine heavy chain constant region (i.e., CH1, CH2, and CH3). In some embodiments, the capture antibody, the detection antibody, or both are chimeric (e.g., comprise at least one constant region or Fc domain that is derived from a different species, such as a murine antibody with a human Fc domain). In some embodiments, the capture antibody and the detection antibody are from the same species. In some embodiments, the capture antibody an IgG. In some embodiments, the detection antibody an IgG.

In some embodiments, the detection antibody comprises a detectable label, such as a fluorophore, enzyme, chemiluminescent agent, or the like. In some embodiments, the detectable label is directly attached to the detection antibody. In some embodiments, the detectable label is attached to the detection antibody via a linker, which may be a chemical linker, a peptide linker, or a combination thereof. In some such embodiments, use of a labeled detection antibody facilitates direct detection of a target of interest.

In some embodiments, the detection antibody does not comprise a label. In some such embodiments, the method further comprises applying a secondary antibody (e.g., an antibody that specifically binds to a detection antibody). In some such embodiments, the secondary antibody further comprises a detectable label. In some embodiments, use of a detection antibody and a secondary antibody facilitates indirect detection of a target of interest. In some embodiments, the secondary antibody specifically binds the detection antibody (and not the capture antibody). In some embodiments, the detection antibody and the secondary antibody are from the different species. For example, the detection antibody may be a human or humanized IgG antibody, comprise a human IgG constant region, or comprises a human IgG Fc domain, while the secondary antibody is a non-human (e.g., a murine, rat, goat, etc.) antibody that specifically binds to a human IgG constant region or Fc domain. In an alternative example, the detection antibody may be a murine IgG antibody, comprise a murine IgG constant region, or comprises a murine IgG Fc domain, while the secondary antibody is a non-murine (e.g., a human, rat, goat, etc.) antibody that specifically binds to a murine IgG constant region or Fc domain. In some embodiments, the capture antibody and the detection antibody are from different species (e.g., human, mouse, rabbit) or comprise constant regions or Fc domains that are from different species, which may be advantageous to reduce and/or eliminate cross-reactivity between the secondary antibody and the capture antibody.

In some embodiments, the methods may further comprise a wash step(s) between one or more of the foregoing steps of the method (e.g., addition of capture antibody, blocking, contacting of sample, addition of detection antibody, addition of secondary antibody) to remove non-bound and/or low-affinity bound non-target components of the sample. Without wishing to be bound by any one theory, removal of non-bound and/or low-affinity bound non-target components of the sample decreases background signal, thereby increasing the signal-to-noise ratio.

In some embodiments, ELISA assays described herein detect and/or quantify dsRNA with improved sensitivity and/or specificity relative to an appropriate reference (e.g., a different method of dsRNA detection and/or quantification, such as a dot blot). In some embodiments, ELISA assays described herein detect dsRNA with increased sensitivity by a factor of about 1.5, 2, 3, 4, 5, 6, 7, 8, 9, 10, 15, 20, 25, 50, 75, or 100 times relative to an appropriate reference.

In some embodiments, technologies of the present disclosure can be used in multiplex assays. In some embodiments, multiplex ELISA comprises coating multiple specific capture antibodies (e.g., including at least one capture antibodies or antigen-binding polypeptides described herein) at multiple spots (one antibody in one spot) in the same well in a multi-well plate. Detectable labels can then employed to detect multiple targets of interest (e.g., dsRNA) at the corresponding spots on the plate.

### Targets and Samples

Technologies of the present disclosure, among other things, can be utilized in methods for detecting dsRNAs in a sample. In some embodiments, a sample refers to an aliquot of material obtained or derived from a source of interest. In some embodiments, a source of interest is or comprises the product of an in vitro transcription reaction. In some embodiments, a source of interest is or comprises drug substance of a RNA-based therapeutic (e.g., mRNA-based therapeutic). In some embodiments, a source of interest is or comprises drug product of a RNA-based therapeutic (e.g., mRNA-based therapeutic).

In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual.

In some embodiments, a sample is or comprises a crude sample obtained directly from a source of interest by any appropriate means. In some embodiments, as will be clear from context, the term sample refers to a preparation that is obtained by processing (e.g., by removing one or more components of and/or by adding one or more agents to a crude sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

In some embodiments, a sample is diluted prior to use in methods of detection and/or quantifying dsRNA as described elsewhere herein. In some embodiments, a sample is diluted by at least about 1:2, at least about 1:3, at least about 1;4, at least about 1:5, at least about 1:10, at least about 1:20, at least about 1:30, at least about 1:40, at least about 1:50, at least about 1:100, at least about 1:150, at least about 1:200, at least about 1:250, at least about 1:300, at least about 1:350, at least about 1:400, at least about 1:450, at least about 1:500, at least about 1:1,000, at least about 1:2,000, at least about 1:3,000, at least about 1:4,000, at least about 1:5,000, at least about 1:10,000, at least about 1:20,000, at least about 1:30,000, at least about 1:40,000, at least about 1;50,000, or at least about 1:100,000. Any dilution buffer commonly used under these circumstances known in the art may be used. Exemplary dilution buffers include, without limitation, phosphate buffered saline (PBS), PBS with Tween20 (PBST), saline, tris buffered saline, HEPES buffer, etc.

### Kits

The present disclosure provides a kit comprising one or more of (a) an anti-dsRNA antibody or antigen-binding polypeptide described herein; and (b) one or more reagents and/or samples for use in accordance with methods of detection and/or quantifying dsRNA (e.g., ELISA assays described herein). In some embodiments, a kit may further comprise instructions for a method (e.g., ELISA assay described herein) of detecting and/or quantifying dsRNA in a sample by an anti-dsRNA antibody or antigen-binding polypeptide described herein.

For the purposes of the present disclosure, a kit will generally comprise at least a capture antibody that binds to dsRNA antibody and a detection antibody that binds to dsRNA. As described above with respect to the disclosed methods, the capture antibody and the detection antibody may comprise Fc domains or constant regions that are from different species. For example, the capture antibody may comprise a mouse Fc domain (or constant region) while the detection antibody comprises a human Fc domain (or constant region), or, alternatively, the capture antibody may comprise a human Fc domain (or constant region) while the detection antibody comprises a murine Fc domain (or constant region). Further, the capture antibody and the detection antibody comprise the same CDRs or variable regions (e.g., VH/VL) even though these antibodies comprise an Fc domain or constant region from different species. In some embodiments, the capture antibody, detection antibody, or both may be an IgG.

In some embodiments, the capture antibody may comprise (i) heavy chain CDRs of SEQ ID NO: 3 and light chain CDRs of SEQ ID NO: 9, or (ii) heavy chain CDRs of SEQ ID NO: 4 and light chain CDRs of SEQ ID NO: 10. In some embodiments, the detection antibody may comprise (i) heavy chain CDRs of SEQ ID NO: 3 and light chain CDRs of SEQ ID NO: 9, or (ii) heavy chain CDRs of SEQ ID NO: 4 and light chain CDRs of SEQ ID NO: 10.

As described above, the detection antibody may comprise a detectable label, which may be directly attached to the antibody or attached indirectly via a linker. Alternatively, a kit may comprise a secondary antibody that binds to the detection antibody and which comprises a detectable label. The detectable label is not particularly limited and may be, for example, a fluorophore, enzyme, chemiluminsecent agent or the like. In some embodiments, the detectable label can be horse radish peroxidase (HRP) or a fluorophore. If an enzyme, such as HRP is utilized as the detectable label, the kit may further comprise a substrate for the enzyme (e.g., a chromogenic or chemiluminescent substrate).

The disclosed kits may further comprise one or more additional reagents for detecting dsRNA, including but not limited to, a blocking buffer, one or more wash buffer(s), a positive control, or a combination thereof.

In some embodiments, a sample for use in accordance with a detection assay is or comprises the product of an in vitro transcription reaction. In some embodiments, a sample for use in accordance with a detection assay is or comprises drug substance of a RNA-based therapeutic (e.g., mRNA-based therapeutic). In some embodiments, a sample for use in accordance with a detection assay is or comprises drug product of a RNA-based therapeutic (e.g., mRNA-based therapeutic).

### EXAMPLES

### A. Example 1: Detection of dsRNA in a Sample

The present example demonstrates detection of dsRNA in a sample using exemplary antibodies, Ab1, Ab2, Ab3, and Ab4, as capture antibodies. A first control anti-dsRNA antibody, Ab5, was utilized as a positive control capture antibody. Briefly, diluted capture antibody was added to microplate wells and incubated. Following incubation, the microplate wells were washed and blocking buffer was added to each microplate well and incubated.

An assay control was prepared and a 400 base pair (bp) calibration standard was prepared and each were serially diluted. Test samples were thawed and serial dilutions were prepared.

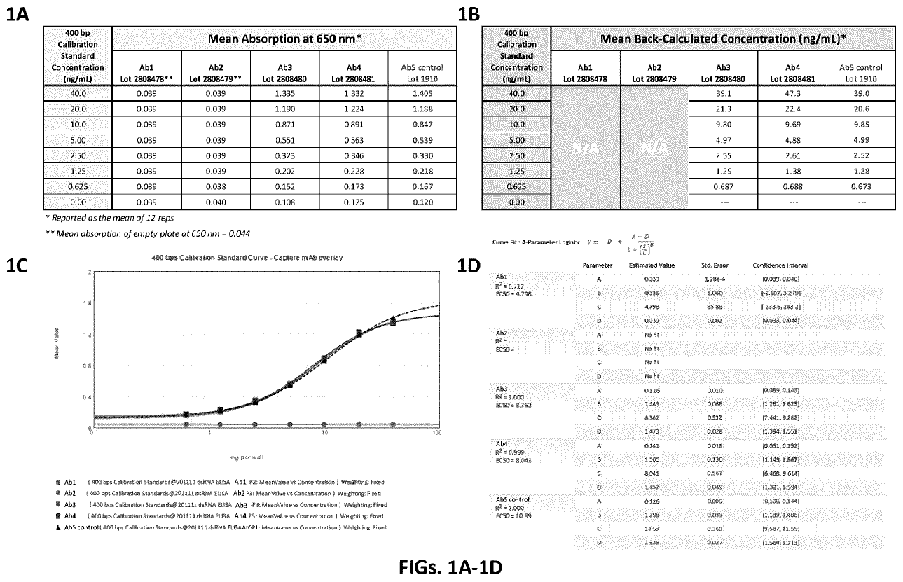

Microplate wells coated with capture antibody were washed and calibration standards, assay controls, and diluted test samples were added in triplicate to a subset of wells in the microplate and incubated. Following incubation, the microplate wells were washed and diluted detection antibody, a second control anti-dsRNA antibody, mouse IgM mAb (14× dilution), was added to each well. The microplate comprising detection antibody was incubated, washed, and secondary antibody, goat anti-mouse IgM-HRP conjugate (0.040 μg/mL) was added to each well. The microplate was incubated, washed, and an HRP substrate (TMB) was added to each well. Color was allowed to develop for approximately 10 minutes until the reaction was stopped by adding stop solution to each well. Absorbance was read using a plate reader. The mean absorption of Ab1, Ab2, Ab3, and Ab4 is shown in FIG. 1A. Ab1 and Ab2 did not detect dsRNA, while Ab3 and Ab4 detected dsRNA with an EC₅₀ of 8.362 and 8.041, respectively. Control anti-dsRNA antibody, Ab5, detected dsRNA with an EC₅₀ of 10.59 (FIG. 1B-1D).

1. An antibody or antigen-binding fragment thereof that binds to double stranded RNA (dsRNA), wherein the antibody or antigen-binding fragment thereof comprises:
(i) complementarity determining regions (CDRs) of a heavy chain variable domain comprising an amino acid sequence selected from SEQ ID NO: 1, SEQ ID NO: 2, SEQ ID NO: 3, and SEQ ID NO: 4; and
(ii) CDRs of a light chain variable domain comprising an amino acid sequence selected from SEQ ID NO: 9 and SEQ ID NO: 10.

2. The antibody or antigen-binding fragment thereof according to claim 1, wherein the antibody or antigen-binding fragment thereof comprises the CDRs of the heavy chain variable domain comprising the amino acid sequence of SEQ ID NO: 1 and the CDRs of the light chain variable domain comprising the amino acid sequence of SEQ ID NO: 9.

3. The antibody or antigen-binding fragment thereof according to claim 1, wherein the antibody or antigen-binding fragment thereof comprises the CDRs of the heavy chain variable domain comprising the amino acid sequence of SEQ ID NO: 2 and the CDRs of the light chain variable domain comprising the amino acid sequence of SEQ ID NO: 9.

4. The antibody or antigen-binding fragment thereof according to claim 1, wherein the antibody or antigen-binding fragment thereof comprises the CDRs of the heavy chain variable domain comprising the amino acid sequence of SEQ ID NO: 3 and the CDRs of the light chain variable domain comprising the amino acid sequence of SEQ ID NO: 10.

5. The antibody or antigen-binding fragment thereof according to claim 1, wherein the antibody or antigen-binding fragment thereof comprises the CDRs of the heavy chain variable domain comprising the amino acid sequence of SEQ ID NO: 4 and the CDRs of the light chain variable domain comprising the amino acid sequence of SEQ ID NO: 10.

6. The antibody or antigen-binding fragment thereof according to claim 1, wherein the antibody or antigen-binding fragment thereof comprises mammalian framework regions and mammalian constant regions.

7. The antibody or antigen-binding fragment thereof according to claim 1, wherein the antibody is an IgG antibody.

8. The antibody or antigen-binding fragment thereof according to claim 1, wherein the antibody or antigen-binding fragment thereof comprises a heavy chain variable domain comprising an amino acid sequence selected from the group consisting of SEQ ID NO: 1, SEQ ID NO: 2, SEQ ID NO: 3, and SEQ ID NO: 4.

9. The antibody or antigen-binding fragment thereof according to claim 1, wherein the antibody or antigen-binding fragment thereof comprises a light chain variable domain comprising an amino acid sequence selected from the group consisting of SEQ ID NO: 9 and SEQ ID NO: 10.

10. The antibody or antigen-binding fragment thereof according to claim 1, wherein the antibody or antigen-binding fragment thereof comprises a heavy chain comprising an amino acid sequence selected from the group consisting of SEQ ID NO: 5, SEQ ID NO:6, SEQ ID NO: 7, and SEQ ID NO: 8 and a light chain comprising an amino acid sequence selected from the group consisting of SEQ ID NO: 11 and SEQ ID NO: 12.

11. The antibody or antigen-binding fragment thereof according to claim 10, wherein the heavy chain comprises the amino acid sequence of SEQ ID NO: 5, and the light chain comprises the amino acid sequence of SEQ ID NO: 11.

12. The antibody or antigen-binding fragment thereof according to claim 10, wherein the heavy chain comprises the amino acid sequence of SEQ ID NO: 6, and the light chain comprises the amino acid sequence of SEQ ID NO: 11.

13. The antibody or antigen-binding fragment thereof according to claim 10, wherein the heavy chain comprises the amino acid sequence of SEQ ID NO: 7, and the light chain comprises the amino acid sequence of SEQ ID NO: 12.

14. The antibody or antigen-binding fragment thereof according to claim 10, wherein the heavy chain comprises the amino acid sequence of SEQ ID NO: 8, and the light chain comprises the amino acid sequence of SEQ ID NO: 12.

15. A method for detecting or quantifying double stranded RNA (dsRNA) in a sample comprising:
(i) contacting the sample with a capture antibody comprising an anti-dsRNA antibody or antigen-binding fragment thereof according to claim 1, thereby producing a first complex comprising the dsRNA and the capture antibody, wherein the capture antibody is immobilized on a solid substrate;
(ii) contacting the first complex with a detection antibody comprising an anti-dsRNA antibody or antigen-binding fragment thereof according to claim 1, thereby producing a second complex comprising the dsRNA, the capture antibody, and the detection antibody;
(iii) optionally contacting the second complex with a secondary antibody that binds to the detection antibody;
wherein either of the detection antibody or the secondary antibody comprises a label capable of producing a signal; and
(iv) measuring the signal.

16. A method for detecting or quantifying double stranded RNA (dsRNA) in a sample comprising:
(i) contacting the sample with a capture antibody that binds to dsRNA, thereby producing a first complex comprising the dsRNA and the capture antibody, wherein the capture antibody is immobilized on a solid substrate;
(ii) contacting the first complex with a detection antibody that binds to dsRNA, thereby producing a second complex comprising the dsRNA, the capture antibody, and the detection antibody;
(iii) optionally contacting the second complex with a secondary antibody that binds to the detection antibody;
wherein either of the detection antibody or the secondary antibody comprises a label capable of producing a signal; and
(iv) measuring a signal;
wherein the capture antibody and the detection antibody comprise the same CDRs but comprise different Fc domains or constant regions.

17. A kit comprising a capture antibody that binds to an anti-dsRNA antibody and a detection antibody that binds to dsRNA.

18. The kit of claim 17, wherein the capture antibody and the detection antibody comprise Fc domains or constant regions that are from different species.

19. The kit of claim 17, wherein the capture antibody comprises (i) heavy chain CDRs of SEQ ID NO: 3 and light chain CDRs of SEQ ID NO: 9, or (ii) heavy chain CDRs of SEQ ID NO: 4 and light chain CDRs of SEQ ID NO: 10.

20. The kit of claim 17, wherein the detection antibody comprises (i) heavy chain CDRs of SEQ ID NO: 3 and light chain CDRs of SEQ ID NO: 9, or (ii) heavy chain CDRs of SEQ ID NO: 4 and light chain CDRs of SEQ ID NO: 10.
